(19) United States
(12) Patent Application Publication
(10) Pub. No.: US 2024/0076412 A1
(43) Pub. Date: Mar. 7, 2024

(54) A BISPECIFIC ANTIBODY TARGETING GPC3 AND CD47

(71) Applicant: NATIONAL INSTITUTE OF BIOLOGICAL SCIENCES, BEIJING, Beijing (CN)

(72) Inventors: Jianhua SUI, Beijing (CN); Kaixin DU, Beijing (CN); Wei CHEN, Beijing (CN); Yulu LI, Beijing (CN); Juan LIU, Beijing (CN)

(21) Appl. No.: 18/270,808

(22) PCT Filed: Jan. 5, 2022
(86) PCT No.: PCT/CN2022/070361

§ 371 Date: Jul. 3, 2023

(30) Foreign Application Priority Data
Jan. 5, 2021 (WO) PCT/CN2021/070378

(51) Int. Cl. C07K 16/46; A61P 35/00

(52) U.S. Cl. CPC C07K 16/468; A61P 35/00; C07K 2317/31; C07K 2317/522; C07K 2317/524; C07K 2317/526; C07K 2317/53; C07K 2317/565

(57) ABSTRACT

An isolated anti-CD47 antibody or fragment thereof has the ability of binding CD47 and competing with the binding of SIRPa to CD47, and comprises a heavy chain variable region comprising heavy chain complementarity determining regions HCDR1, HCDR2, and HCDR3; and a light chain variable region comprising light chain complementarity determining regions LCDR1, LCDR2, and LCDR3. A method for treating a disorder in which CD47 is overexpressed or upregulated in a subject, comprises using an isolated anti-CD47 antibody or fragment thereof having the ability of binding CD47 and competing with the binding of SIRPa to CD47. A bispecific antibody comprises a first antigen binding moiety that binds to human GPC3 (hGPC3); and a second antigen binding moiety that binds to human CD47 (hCD47).

### INTRODUCTION

Hepatocellular carcinoma (HCC) is the sixth most common carcinoma worldwide and the third leading cause of cancer-related death. For patients with de novo or recurrent advanced HCC, the treatment options are limited. Currently, only two multikinase inhibitors—Sorafenib and Lentivanib—have been approved by the FDA as first-line systemic treatments for advanced HCC. However, their treatment benefits and response rates are often modest. Tumor antigen-targeting antibody- and immune modulating antibody-based immunotherapies represent emerging approaches that may improve HCC treatment outcomes.

Glypican-3 (GPC3) is one of the best-characterized HCC-associated antigens. It is a member of the family of glypicans, that are heparin sulfate proteoglycans anchored to cell membranes by a glycosylphosphatidylinositol (GPI) anchor. Many studies have confirmed that GPC3 is specifically up-regulated in HCC, with minimal or no expression in normal (and even cirrhotic) liver tissues, making it an excellent tumor-specific target for an antibody therapy. However, the humanized anti-GPC3 antibody, for example, GC33, did not confer a clinical benefit in a phase II clinical trial as a monotherapy, potentially owing to suboptimal dosing and/or an unfavorable immune environment.

CD47 is an inhibitory innate immune checkpoint. It interacts with its receptor signal regulatory protein alpha (SIRPα) on myeloid cells (especially on macrophages) and confers a “don't eat me” signal so that cancer cells can evade immune surveillance. Therefore, blockade of the interaction between CD47 and SIRPα with antibodies targeting CD47 represents a promising strategy to enhance the phagocytic clearance of cancer cells. The expression of CD47 is up-regulated in many solid tumors including HCC, and anti-CD47 antibodies can inhibit HCC tumor growth. However, CD47-targeted antibodies that are currently under clinical development are cleared rapidly and lead to hemotoxicity, owing largely to the expression of CD47 on normal cells, and especially on red blood cells.

### SUMMARY OF THE INVENTION

In the first place, the present invention relates to an isolated anti-CD47 antibody or fragment thereof having the ability of binding CD47 and competing with the binding of SIRPa to CD47.

In some embodiments, the anti-CD47 antibody or fragment thereof according to the present invention is capable of binding CD47. Preferably, the anti-CD47 antibody or fragment thereof according to the present invention binds to human CD47 (hCD47).

In some embodiments, the anti-CD47 antibody or fragment thereof according to the present invention is a human anti-CD47 antibody.

In some embodiments, the anti-CD47 antibody or fragment thereof according to the present invention is a monoclonal antibody.

In some embodiments, the anti-CD47 antibody or fragment thereof according to the present invention binds to CD47 with high affinity, with a dissociation constant (KD) of less than about 10 nM, e.g., less than 4 nM. In some embodiments, the anti-CD47 antibody or fragment thereof according to the present invention is capable of binding to hCD47 on cell surface, for example, binding to hCD47 on cell surface of Raji and HEK-293 even at low antibody concentration of 0.06 nM.

In some embodiments, the anti-hCD47 antibody or fragment thereof according to the present invention is capable of blocking CD47/SIRPα interaction, and enables phagocytosis of target cells.

In some embodiments, the anti-hCD47 antibody or fragment thereof according to the present invention is capable of inducing potent macrophage-mediated phagocytosis of HL-60.

In some embodiments, the anti-hCD47 antibody or fragment thereof according to the present invention is capable of reducing or even curing human lymphoma or breast cancer. For example, the anti-hCD47 antibody or fragment thereof according to the present invention is capable of exerting anti-tumor effect against human lymphoma in immunodeficient mice. For example, the anti-hCD47 antibody or fragment thereof according to the present invention is capable of exerting anti-tumor effect against human breast cancer in immunodeficient mice.

In some embodiments, it is shown that big tumors in NSG mice engrafted with Raji are shrunk with the anti-hCD47 antibody or fragment thereof according to the present invention.

The anti-hCD47 antibody or fragment thereof according to the present invention, in the non-specific binding experiment, shows no binding activity to CHO, Hepa1-6, B16F10, BHK-21, CT26 cell lines that do not expressed human CD47 even at high antibody concentration of 500 nM.

The anti-hCD47 antibody or fragment thereof according to the present invention shows good thermal stability.

In some embodiments, the anti-hCD47 antibody or fragment thereof according to the present invention is capable of binding to soluble forms of hCD47. Preferably, the anti-hCD47 antibody is capable of binding to the soluble form of the extracellular domain of hCD47.

In some embodiments, the anti-hCD47 antibody or fragment thereof according to the present invention is capable of binding to residues 1-118 of the extracellular domain of the human CD47.

In some embodiments, the anti-CD47 antibody or fragment thereof according to the present invention comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:
HCDR1, HCDR2, and HCDR3 are selected from the group consisting of: (1) HCDR1 having the amino acid sequence of SEQ ID NO: 1, HCDR2 having the amino acid sequence of SEQ ID NO: 2, HCDR3 having the amino acid sequence of SEQ ID NO: 3; (2) HCDR1 having the amino acid sequence of SEQ ID NO: 4, HCDR2 having the amino acid sequence of SEQ ID NO: 5, HCDR3 having the amino acid sequence of SEQ ID NO: 6; (3) HCDR1 having the amino acid sequence of SEQ ID NO: 7, HCDR2 having the amino acid sequence of SEQ ID NO: 8, HCDR3 having the amino acid sequence of SEQ ID NO: 9; (4) HCDR1 having the amino acid sequence of SEQ ID NO: 10, HCDR2 having the amino acid sequence of SEQ ID NO: 11, HCDR3 having the amino acid sequence of SEQ ID NO: 12; (5) HCDR1 having the amino acid sequence of SEQ ID NO: 13, HCDR2 having the amino acid sequence of SEQ ID NO: 14, HCDR3 having the amino acid sequence of SEQ ID NO: 15; and (6) HCDR1, HCDR2, HCDR3 as shown in (1)-(5), but at least one of which includes one, two, three, four or five amino acids addition, deletion, conservative amino acid substitution or the combinations thereof; and LCDR1, LCDR2, and LCDR3 are selected from the group consisting of:
(1) LCDR1 having the amino acid sequence of SEQ ID NO: 16, LCDR2 having the amino acid sequence of SEQ ID NO: 17, LCDR3 having the amino acid sequence of SEQ ID NO: 18; (2) LCDR1 having the amino acid sequence of SEQ ID NO: 19, LCDR2 having the amino acid sequence of SEQ ID NO: 20, LCDR3 having the amino acid sequence of SEQ ID NO: 21; (3) LCDR1 having the amino acid sequence of SEQ ID NO: 22, LCDR2 having the amino acid sequence of SEQ ID NO: 23, LCDR3 having the amino acid sequence of SEQ ID NO: 24; (4) LCDR1 having the amino acid sequence of SEQ ID NO: 25, LCDR2 having the amino acid sequence of SEQ ID NO: 26, LCDR3 having the amino acid sequence of SEQ ID NO: 27; (5) LCDR1 having the amino acid sequence of SEQ ID NO: 28, LCDR2 having the amino acid sequence of SEQ ID NO: 29, LCDR3 having the amino acid sequence of SEQ ID NO: 30; and (6) LCDR1, LCDR2, LCDR3 as shown in (1)-(5), but at least one of which includes one, two, three, four or five amino acids addition, deletion, conservative amino acid substitution or the combinations thereof.

In one embodiment, the anti-CD47 antibody or fragment thereof according to the present invention comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:
HCDR1, HCDR2, HCDR3, LCDR1, LCDR2, and LCDR3 are selected from the group consisting of: (1) HCDR1 having the amino acid sequence of SEQ ID NO: 1, HCDR2 having the amino acid sequence of SEQ ID NO: 2, HCDR3 having the amino acid sequence of SEQ ID NO: 3, LCDR1 having the amino acid sequence of SEQ ID NO: 16, LCDR2 having the amino acid sequence of SEQ ID NO: 17, LCDR3 having the amino acid sequence of SEQ ID NO: 18; (2) HCDR1 having the amino acid sequence of SEQ ID NO: 4, HCDR2 having the amino acid sequence of SEQ ID NO: 5, HCDR3 having the amino acid sequence of SEQ ID NO: 6, LCDR1 having the amino acid sequence of SEQ ID NO: 19, LCDR2 having the amino acid sequence of SEQ ID NO: 20, LCDR3 having the amino acid sequence of SEQ ID NO: 21; (3) HCDR1 having the amino acid sequence of SEQ ID NO: 7, HCDR2 having the amino acid sequence of SEQ ID NO: 8, HCDR3 having the amino acid sequence of SEQ ID NO: 9, LCDR1 having the amino acid sequence of SEQ ID NO: 22, LCDR2 having the amino acid sequence of SEQ ID NO: 23, LCDR3 having the amino acid sequence of SEQ ID NO: 24; (4) HCDR1 having the amino acid sequence of SEQ ID NO: 10, HCDR2 having the amino acid sequence of SEQ ID NO: 11, HCDR3 having the amino acid sequence of SEQ ID NO: 12, LCDR1 having the amino acid sequence of SEQ ID NO: 25, LCDR2 having the amino acid sequence of SEQ ID NO: 26, LCDR3 having the amino acid sequence of SEQ ID NO: 27; (5) HCDR1 having the amino acid sequence of SEQ ID NO: 13, HCDR2 having the amino acid sequence of SEQ ID NO: 14, HCDR3 having the amino acid sequence of SEQ ID NO: 15, LCDR1 having the amino acid sequence of SEQ ID NO: 28, LCDR2 having the amino acid sequence of SEQ ID NO: 29, LCDR3 having the amino acid sequence of SEQ ID NO: 30; and (6) HCDR1, HCDR2, HCDR3, LCDR1, LCDR2, LCDR3 as shown in (1)-(5), but at least one of which includes one, two, three, four or five amino acids addition, deletion, conservative amino acid substitution or the combinations thereof.

In some embodiments, the anti-CD47 antibody or fragment thereof according to the present invention comprises a heavy chain variable region, and a light chain variable region, wherein the heavy chain variable region has the amino acid sequence selected from the group consisting of the amino acid sequences shown in SEQ ID NOs: 37-41, and an amino acid sequence having at least 95% sequence identity to any one of the amino acid sequences shown in SEQ ID NOs: 37-41, and retaining the activity of epitope-binding,

wherein the light chain variable region has the amino acid sequence selected from the group consisting of the amino acid sequences shown in SEQ ID NOs: 42-46, or an amino acid sequence having at least 95% sequence identity to any one of the amino acid sequences shown in SEQ ID NOs: 42-46, and retaining the activity of epitope-binding.

In some embodiments, the anti-CD47 antibody or fragment thereof according to the present invention comprises a heavy chain variable region, and a light chain variable region, wherein the heavy chain variable region and the light chain variable region have the amino acid sequences selected from the group consisting of:
(1) the amino acid sequence shown in SEQ ID NO: 37, and the amino acid sequence shown in SEQ ID NO: 42; (2) the amino acid sequence shown in SEQ ID NO: 38, and the amino acid sequence shown in SEQ ID NO: 43; (3) the amino acid sequence shown in SEQ ID NO: 39, and the amino acid sequence shown in SEQ ID NO: 44; (4) the amino acid sequence shown in SEQ ID NO: 40, and the amino acid sequence shown in SEQ ID NO: 45; (5) the amino acid sequence shown in SEQ ID NO: 41, and the amino acid sequence shown in SEQ ID NO: 46; and (6) two amino acid sequences having at least 95% sequence identity to any one of (1)-(5) respectively, and retaining the activity of epitope-binding.

In some embodiments, the anti-CD47 antibody or fragment thereof according to the present invention is an isotype of IgG, IgM, IgA, IgE or IgD. In some embodiments, the anti-CD47 antibody or fragment thereof according to the present invention is an isotype of IgG1, IgG2, IgG3, or IgG4.

Preferably, the monoclonal antibody (mAb) of the present invention is human monoclonal antibody (mAb).

In some embodiments, the polynucleotide according to the present invention may encode the entire heavy chain variable region, or the entire light chain variable region, or the both on the same polynucleotide molecule or on separate polynucleotide molecules. Alternatively, the polynucleotide according to the present invention may encode portions of heavy chain variable region, or the light chain variable region, or the both on the same polynucleotide molecule or on separate polynucleotide molecules.

In another aspect, the present invention provides an isolated cell, or vector comprising one or more polynucleotide encoding the anti-CD47 antibody or fragment thereof according to the present invention.

In another aspect, the present invention provides a composition comprising the anti-CD47 antibody or fragment thereof according to the present invention and a pharmaceutical acceptable carrier.

In another aspect, the present invention provides use of the anti-CD47 antibody or fragment thereof according to the present invention in manufacturing a medicament for treating a disorder in which CD47 is overexpressed or upregulated in a subject.

The subject may be a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

In some embodiments, the disorder is a cancer including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, etc.) and liquid cancers (e.g., hematological cancers, leukemias, lymphomas, etc.) and brain cancers.

In some embodiments, the disorder is an infection (e.g., chronic infection); and/or an immunological disease or disorder (e.g., an inflammatory disease, including but not limited to multiple sclerosis, arthritis, and the like).

In another aspect, the present invention provides a method for treating a disorder, in which CD47 is overexpressed or upregulated, in a subject, comprising administering to the patient the anti-CD47 antibody or fragment thereof according to the present invention.

The subject may be a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

In another aspect, the present invention provides a method for determining the presence of CD47, comprising exposing a cell suspected of containing CD47 to the anti-CD47 antibody or fragment thereof according to the present invention, and determining binding of the anti-CD47 antibody or fragment thereof to the cell.

The method may be a method for diagnosing a disorder, in which CD47 is overexpressed or upregulated, in a subject.

The subject may be a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

In the second place, the present invention relates to a bispecific antibody or fragment thereof having the ability of specifically binding to both GPC3 and CD47. Preferably, the bispecific antibody or fragment thereof has the ability of specifically binding to both hGPC3 and hCD47.

In some embodiments, the bispecific antibody according to the present invention is a bispecific antibody comprising a first antigen binding moiety that binds to human GPC3(hGPC3) and a second antigen binding moiety that binds to human CD47(hCD47). Hereafter, the bispecific antibody according to the present invention is also referred to as GPC3/CD47 biAb.

In some embodiments, the first antigen binding moiety that binds to human GPC3 comprises:
(a) a VH domain comprising (i) HCDR1 having the amino acid sequence of SEQ ID NO: 31, (ii) HCDR2 having the amino acid sequence of SEQ ID NO: 32, and (iii) HCDR3 having the amino acid sequence of SEQ ID NO: 33; and (b) a VL domain comprising (i) LCDR1 having the amino acid sequence of SEQ ID NO: 34, (ii) LCDR2 having the amino acid sequence of SEQ ID NO: 35, and (iii) LCDR3 having the amino acid sequence of SEQ ID NO: 36.

In some embodiments, the first antigen binding moiety that binds to human GPC3 comprises:
(a) a VH domain comprising the amino acid sequence of SEQ ID NO: 47; and (b) a VL domain comprising the amino acid sequence of SEQ ID NO: 48.

In some embodiments, the second antigen binding moiety that binds to human CD47 comprises:
(a) a VH domain comprising (i) HCDR1 having the amino acid sequence of SEQ ID NO: 1, (ii) HCDR2 having the amino acid sequence of SEQ ID NO: 2, and (iii) HCDR3 having the amino acid sequence of SEQ ID NO: 3; and (b) a VL domain comprising (i) LCDR1 having the amino acid sequence of SEQ ID NO: 16, (ii) LCDR2 having the amino acid sequence of SEQ ID NO: 17, and (iii) LCDR3 having the amino acid sequence of SEQ ID NO: 18.

In some embodiments, the first antigen binding moiety that binds to human CD47 comprises:

(a) a VH domain comprising the amino acid sequence of SEQ ID NO: 37; and (b) a VL domain comprising the amino acid sequence of SEQ ID NO: 42.

In some embodiments, the bispecific antibody according to the present invention further comprises an Fc region.

In some embodiments, the Fc antibody domain can include a Hinge portion, a CH3 portion and a CH2 portion.

In some embodiments, the Fc antibody domain can further include domains that promote heterodimerization. In some embodiments, the Fc portion can include a knob domain and a hole domain that allow for heterodimerization of the two heavy chains.

In some embodiments, the knob domain and the hole domain are positioned in CH3 portions respectively.

Preferably, the knob domain has the knob mutations, and the hole domain has the hole mutations. Preferably, the knob mutations are T366W and S354C, and the hole mutations are Y407V, L368A, T366S, and cysteine mutations are added to further promote heterodimerization.

In some embodiments, the bispecific antibody according to the present invention has a CH1 portion and a CL portion.

In some embodiments, CH1 portion and a CL portion are replaced by each other.

In some embodiments, the bispecific antibody according to the present invention comprises a knob chain(HC1 chain) and a hole chain(HC2 chain), wherein the knob chain comprises the first antigen binding moiety, CH1 portion, hinge portion, CH2 portion and CH3 region, and the hole chain comprises the second antigen binding moiety, CL portion, Hinge portion, CH2 portion and CH3 portion. Preferably, the bispecific antibody according to the present invention further comprises LC1 portion comprising VL and CL, and LC2 portion comprising VL and CH1.

In some embodiments, the bispecific antibody according to the present invention comprises HC1 having the amino acid sequence of SEQ ID NO: 49, LC1 portion having the amino acid sequence of SEQ ID NO: 50, HC2 having the amino acid sequence of SEQ ID NO: 51, and LC2 portion having the amino acid sequence of SEQ ID NO: 52, and HC1 and HC2 are interlinked by three disulfide bonds and in a knob-into-hole way, and LC1 and LC2 attached to HC1 and HC2 respectively by a disulfide bond.

Specifically, the original wide-type HC and LC expression vectors using previously reported KiHs and CrossMab technologies are modified. Briefly, several mutations were introduced into the CH3 domain of the HC via site-directed mutagenesis to generate the “knob chain” (T366W, S354C) and the “hole chain” (Y349C, T366S, L368A, Y407V). The CH1 domain of the hole chain and the CL domain of the LC were interchanged to respectively generate the paired crossover heavy chain (CL-hole chain) and light chain (CH1-LC).

In some embodiments, the bispecific antibody according to the present invention shows greatly improved specificity for dual antigen-expressing cells (GPC3⁺CD47⁺ double positive cells).

In some embodiments, the bispecific antibody according to the present invention shows superior safety profile and extended serum half-life in human CD47/SIRPα gene-modified mice compared with anti-CD47 mAb.

In some embodiments, the bispecific antibody according to the present invention shows enhanced Fc-mediated functions and selective growth suppression against dual antigen-expressing tumors, for example Raji-GPC3^{H} cells.

In some embodiments, the bispecific antibody according to the present invention outperforms monotherapies using an anti-CD47 or anti-GPC3 mAbs, and an anti-CD47 and anti-GPC3 mAbs combination therapy in a xenograft HCC model.

In the present invention, it is found that macrophages and neutrophils are involved in the bispecific antibody's Fc-dependent antitumor activities in vivo.

In the third place, the present invention relates to use of the bispecific antibody of the second place for treating HCC.

The bispecific antibody according to the present invention binds to GPC3 and CD47 to specifically target HCC tumor cells, shows an attractive half-life and displays no hematological toxicity in the pharmacokinetics and potential adverse effects test.

The safety profile, long half-life, and preferential antitumor activity of the bispecific antibody according to the present invention highlight its apparent advantages compared to combinational therapies that employ an anti-CD47 monoclonal antibody.

The present invention illustrates how the exploitation of CD47 blockade can add an additional pro-phagocytic antitumor response for the treatment of CD47 positive HCC.

In the present invention, it is found that neutrophils are required for the antitumor effects of the bispecific antibody in NOD-SCID mice.

The bispecific antibody according to the present invention may also exploit T cell antitumor activity, and its HCC therapeutic efficacy could potentially be further maximized when deployed as a combination agent alongside other T cell stimulating immune checkpoint inhibitor(s) or chemotherapeutic(s). Future studies administering GPC3/CD47 biAb together with T cell immune checkpoint inhibitors such as anti-PD-1 or anti-CTLA-4 mAbs in immune-competent models are warranted to address their respective contributions.

In addition to exhibiting much stronger antitumor activity than monoclonal antibodies alone, treatment with GPC3/CD47 biAb conferred superior antitumor benefit over a combination therapy comprising two antibodies that separately recognize the GPC3 and CD47 targets. This observation supports that the binding of two antigens using a bispecific antibody may offer improved clinical value through some form of synergism. In sum, the present invention develops a bispecific antibody and demonstrated its safe and efficient antitumor activity for HCC. Ultimately, as learning more about the specific mechanisms of GPC3/CD47 biAb in treating HCC, we may uncover more general trends that can support the development of bispecific antibodies to boost innate immune responses for innovative therapeutic strategies to further improve cancer treatment outcomes.

Some studies have used the strategy of T cell-engaging bispecific antibodies (T-BsAbs) that combine two different antigen binding specificities within a single molecule; such antibodies engage T cells with one arm and engage a tumor antigen with the other arm. In addition to harnessing T cell immune responses to fight malignant cells, increasing evidence is underscoring the impacts of the innate immune system in cancer treatment. For example, studies have reported macrophage recruitment in GPC3-positive HCC patients. Phagocytosis of cancer cells is the major mechanism through which macrophages mediate antitumor activity. Therefore, we speculated that harnessing macrophages may be a promising strategy to improve antitumor efficacy against this type of HCC.

### Definitions

Glypican-3 (GPC3) is one of the best-characterized HCC-associated antigens. It is a member of the family of glypicans, that are heparin sulfate proteoglycans anchored to cell membranes by a glycosylphosphatidylinositol (GPI) anchor. Many studies have confirmed that GPC3 is specifically up-regulated in HCC, with minimal or no expression in normal (and even cirrhotic) liver tissues, making it an excellent tumor-specific target for an antibody therapy. The known humanized anti-GPC3 antibodies, for example, GC33, did not confer a clinical benefit in a phase II clinical trial as a monotherapy, potentially owing to suboptimal dosing and/or an unfavorable immune environment.

CD47 is an inhibitory innate immune checkpoint. It interacts with its receptor signal regulatory protein alpha (SIRPα) on myeloid cells (especially on macrophages) and confers a “don't eat me” signal so that cancer cells can evade immune surveillance. Therefore, blockade of the interaction between CD47 and SIRPα with antibodies targeting CD47 represents a promising strategy to enhance the phagocytic clearance of cancer cells. The expression of CD47 is up-regulated in many solid tumors including HCC, and anti-CD47 antibodies can inhibit HCC tumor growth. However, CD47-targeted antibodies that are currently under clinical development are cleared rapidly and lead to hemotoxicity, owing largely to the expression of CD47 on normal cells, and especially on red blood cells.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The products and methods disclosed herein encompass polypeptides and polynucleotides having the sequences specified, or sequences identical or similar thereto, e.g., sequences having at least about 85% or 95% sequence identity(identical) to the sequence specified. In the context of an amino acid sequence, the term “85% or 95% sequence identity(identical)” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 86%, 87%, 88%, 89%, 90%, 91%, 92%, 93%, 94%, 95%, 96%, 97%, 98% or 99% identity to a reference sequence, e.g., a sequence provided herein.

In the context of nucleotide sequence, the term “85% or 95% sequence identity (identical)” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 86%, 87%, 88%, 89%, 90%, 91%, 92%, 93%, 94%, 95%, 96%, 97%, 98% or 99% identity to a reference sequence, e.g., a sequence provided herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The terms “polypeptide”, “peptide” and “protein” are used interchangeably herein to refer to polymers of amino acids of any length.

The terms “nucleic acid”, “nucleic acid sequence”, “nucleotide sequence”, “polynucleotide sequence”, and “polynucleotide” are used interchangeably.

As used herein, the term “antibody or antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain. As used herein, an antibody molecule “binds to” an antigen as such binding is understood by one skilled in the art. In one embodiment, an antibody binds to an antigen with a dissociation constant (KD) of about 1×10⁻⁵ M or less, 1×10⁻⁶ M or less, or 1×10⁻⁷ M or less.

For example, an antibody molecule can include a heavy (H) chain variable domain sequence (abbreviated herein as VH), and a light (L) chain variable domain sequence (abbreviated herein as VL). In an embodiment, an antibody molecule comprises or consists of a heavy chain and a light chain. In another example, an antibody molecule includes two heavy (H) chain variable domain sequences and two light (L) chain variable domain sequences, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

The terms “antibody fragment” or “antigen-binding fragment”, as used herein, is a portion of an antibody such as F(ab′)₂, F(ab)₂, Fab′, Fab, Fv, scFv and the like. An antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

Examples of antigen-binding fragments of an antibody molecule include: (i) a Fab fragment, a monovalent fragment consisting of the VL, VH, CK and CH portions; (ii) a F(ab′)₂ fragment, a bivalent fragment comprising two Fab fragments linked by a disulfide bridge at the hinge region; (iii) a Fd fragment consisting of the VH and CH portions; (iv) a Fv fragment consisting of the VL and VH portions of a single arm of an antibody, (v) a diabody (dAb) fragment, which consists of a VH portion; (vi) a camelid or camelized variable portion; (vii) a single chain Fv(scFv); (viii) a single portion antibody. These antibody fragments may be obtained using any suitable method, including conventional techniques known to those with skill in the art, and the fragments can be screened for utility in the same manner as are intact antibodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. ScFv molecules are known in the art.

The light and heavy chains are divided into regions of “constant” and “variable”. The variable domains of both the light (VL) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. The N-terminal portion is a variable region and at the C-terminal portion is a constant region; the CH3 and CK portions actually comprise the carboxy-terminus of the heavy and light chain, respectively.

The variable region allows the antibody to selectively recognize and specifically bind epitopes on antigens. The VL portion and VH portion, or subset of the complementarity determining regions (CDRs), of an antibody combine to form the variable region that defines a three dimensional antigen-binding site. This quaternary antibody structure forms the antigen-binding site present at the end of each arm of Y. More specifically, the antigen-binding site is defined by three CDRs on each of the VH and VK chains (i.e. HCDR1, HCDR2, HCDR3, LCDR1, LCDR2 and LCDR3).

The terms “complementarity determining region” and “CDR” as used herein refer to the sequences of amino acids within antibody variable regions which confer antigen specificity and binding affinity. In some embodiments, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR can be determined using any of well-known schemes, including those described by Kabat et al. (1991), “Sequences of Proteins of Immunological Interest,” 5th Ed. Public Health Service, National Institutes of Health, Bethesda, MD (“Kabat” numbering scheme).

Each VH and VL typically includes three CDRs and four FRs, arranged from amino-terminus to carboxy-terminus in the following order: FR1, CDR1, FR2, CDR2, FR3, CDR3, FR4.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

As used herein, the term “epitope” refers to the moieties of an antigen (e.g., human GPC3(hGCP3) and human CD47(hCD47)) that specifically interact with an antibody molecule. Such moieties, also referred to herein as epitopic determinants, typically comprise, or are part of, elements such as amino acid side chains or sugar side chains. An epitopic determinant can be defined by methods known in the art or disclosed herein, e.g., by crystallography or by hydrogen-deuterium exchange. At least one or some of the moieties on the antibody molecule that specifically interact with an epitopic determinant are typically located in a CDR(s). Typically, an epitope has a specific three dimensional structural characteristics. Typically, an epitope has specific charge characteristics. Some epitopes are linear epitopes while others are conformational epitopes.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., library selection, and screening, or recombinant methods).

The antibody molecule can be a polyclonal or a monoclonal antibody. In other embodiments, the antibody can be recombinantly produced, e.g., produced by yeast display, phage display, or by combinatorial methods.

In one embodiment, the antibody is a fully human antibody (e.g., an antibody produced by yeast display, an antibody produced by phage display, or an antibody made in a mouse which has been genetically engineered to produce an antibody from a human immunoglobulin sequence), or a non-human antibody, e.g., a murine (mouse or rat), goat, primate (e.g., monkey), or camel antibody. Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein.

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4.

Methods for altering an antibody constant region are known in the art. Antibodies with altered function, e.g. altered affinity for an effector ligand, such as FcR on a cell, or the CI component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Amino acid mutations which stabilize antibody structure, such as S228P (Eu numbering) in human IgG4, are also contemplated.

It is understood that the molecules of the invention may have additional conservative or nonessential amino acid substitutions, which do not have a substantial effect on their functions.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Conservative amino acid substitutions:

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

### BRIEF DESCRIPTION OF THE DRAWINGS

FIG. 1 shows binding kinetics of BC7 and BC18 antibodies to hCD47 by Surface Plasmon Resonance (SPR) analysis.

FIG. 2 shows binding kinetics of BC18, AB6.12, Hu5F9 antibodies to hCD47 by SPR analysis.

FIG. 3 shows comparison of the anti-hCD47 antibodies on the CD47 binding and SIRPα competition abilities by ELISA.

FIG. 4 shows comparison of the anti-hCD47 by FACS analysis.

FIG. 5 shows that BC18 and BC7 bind to cell surface CD47.

FIG. 6 shows that BC18 and BC7 induce potent macrophage-mediated phagocytosis of HL-60.

FIG. 7 shows that BC18 exerts strong anti-tumor effect in human lymphoma model established in immunodeficient mice.

FIG. 8 shows that BC18 exerts strong anti-tumor effect in human breast cancer model established in immunodeficient mice.

FIG. 9 shows binding kinetics of BC18, BC7 and their mutants by SPR analysis.

FIG. 10 shows the schematic structure of the constructed GPC3/CD47 biAb.

FIG. 11 shows construction and characterization of the affinity and purity of purified antibodies.

FIG. 12 shows GPC3/CD47 biAb's greatly improved specificity for dual antigen-expressing cells.

FIG. 13 shows that GPC3/CD47 biAb has a superior safety profile.

FIG. 14 shows that no hematologic toxicity was observed in hCD47/hSIRPα humanized mice after GPC3/CD47 biAb treatment.

FIG. 15 shows generation of CD47 knockout Jurkat-based effector cells (Jurkat-CD16A-CD47^{KO}) and Hep3B cells (Hep3B-CD47^{KO}).

FIG. 16 shows that GPC3/CD47 biAb exerts enhanced Fc-mediated functions and selective growth suppression against dual antigen-expressing tumors.

FIG. 17 shows that GPC3/CD47 biAb outperforms monotherapies and an anti-CD47 and anti-GPC3 mAbs combination therapy in a xenograft HCC model.

FIG. 18 shows that GPC3/CD47 biAb improved median overall survival of Hep3B xenograft mice.

FIG. 19 shows that macrophages and neutrophils are involved in GPC3/CD47 biAb's Fc-dependent antitumor activities in vivo.

FIG. 20 shows generation of the GPC3/CD47 biAb Fc variant (GPC3/CD47-DANG).

FIG. 21 shows analyses of immune cell depletion efficacy and tumor infiltrating macrophages.

### DESCRIPTION OF PARTICULAR EMBODIMENTS OF THE INVENTION

The descriptions of particular embodiments and examples are provided by way of illustration and not by way of limitation. Those skilled in the art will readily recognize a variety of noncritical parameters that could be changed or modified to yield essentially similar results.

TABLE 1

| Name of | | SEQ | Name of | | SEQ |
|---|---|---|---|---|---|
| antibody | Region | ID NO. | antibody | Region | ID NO. |
| BC18m03 | HCDR1 | 1 | BC18m03 | LCDR1 | 16 |
| | HCDR2 | 2 | | LCDR2 | 17 |
| | HCDR3 | 3 | | LCDR3 | 18 |
| BC18 | HCDR1 | 4 | BC18 | LCDR1 | 19 |
| | HCDR2 | 5 | | LCDR2 | 20 |
| | HCDR3 | 6 | | LCDR3 | 21 |
| BC7m03 | HCDR1 | 7 | BC7m03 | LCDR1 | 22 |
| | HCDR2 | 8 | | LCDR2 | 23 |
| | HCDR3 | 9 | | LCDR3 | 24 |
| BC7m04 | HCDR1 | 10 | BC7m04 | LCDR1 | 25 |
| | HCDR2 | 11 | | LCDR2 | 26 |
| | HCDR3 | 12 | | LCDR3 | 27 |
| BC7 | HCDR1 | 13 | BC7 | LCDR1 | 28 |
| | HCDR2 | 14 | | LCDR2 | 29 |
| | HCDR3 | 15 | | LCDR3 | 30 |
| GCP3 | HCDR1 | 31 | GCP3 | LCDR1 | 34 |
| | HCDR2 | 32 | | LCDR2 | 35 |
| | HCDR3 | 33 | | LCDR3 | 36 |
| BC18m03 | VH | 37 | BC18m03 | VL | 42 |
| BC18 | VH | 38 | BC18 | VL | 43 |
| BC7m03 | VH | 39 | BC7m03 | VL | 44 |
| BC7m04 | VH | 40 | BC7m04 | VL | 45 |
| BC7 | VH | 41 | BC7 | VL | 46 |
| GCP3 | VH | 47 | GCP3 | VL | 48 |
| GPC3/CD47 | HC1 | 49 | GPC3/CD47 | LC1 | 50 |
| biAb | | | biAb | | |
| GPC3/CD47 | HC2 | 51 | GPC3/CD47 | HC2 | 52 |
| biAb | | | biAb | | |

### EXAMPLES

### Example 1. Human Monoclonal Antibodies Against the Extracellular of Human CD47 were Generated Based on Phage Display Antibody Technology with Modification

Antigen Purification

The gene fragment encoding the extracellular domain of human CD47 (CD47-ECD), residues 1-118, was obtained from the cDNA of human PBMC and then subcloned into an expression vector containing a His6 tag for purification and Avi tag for Biotinylation. CD47-ECD-His6 with a biotin modification at the C-terminus (CD47-Bio) was expressed by co-transfection 293F cells with the CD47 expressing plasmid and a Biotin ligase (BirA) expression plasmid at 1:1 ratio and purified from cell culture supernatants using Ni-NTA agarose (Qiagen).

Phage Display Antibody Library.

A human non-immune scFv (Single-chain variable fragment) antibody library was constructed from peripheral blood mononuclear cells (PBMCs) of 93 healthy donors. The library has a size of a total of 1.1×10¹⁰ members.

Selection and Screening of Phage Antibody Library

Phage particles expressing scFv on their surface (phage-Ab) were prepared from the library and used for selection of antibodies against the extracellular of human CD47. CD47-Bio was captured on streptavidin-conjugated magnetic M-280 Dynabeads® (Life Technologies) and then incubated with phage particles prepared from the library. Two rounds of selection were performed. For each round of selection, to obtain high-affinity antibodies, the amount of CD47-Bio captured onto the magnetic beads was optimized, and extensive washing steps were applied. Bound phage-Abs were eluted using basic triethanolamine solution. Subsequently, a total of 95 single clones were picked and rescued to produce phage-Abs and screened for specific binding to CD47-Bio by enzyme-linked immunosorbent assay (ELISA). Clones that bound to CD47-Bio with values of optical density at 450 nm>0.2 were scored as positive, whereas negative clones gave values of <0.1. After sequencing the genes of variable regions of heavy (VH) and light (VL) chain of positive clones, their corresponding amino acid sequences were aligned to eliminate repeated clones and identify unique antibodies with a different sequence for further characterization. In the end, five antibodies were identified, they are named BC2, BC7, BC12, BC15, BC18.

SIRPα-d1 Purification

The gene fragment encoding the first domain of human SIRPα (SIRPα-d1), residues 1-148, was amplified from the cDNA of human PBMC. For expressing the SIRPα-d1-mFc fusion protein, the SIRPα-d1 gene was subcloned into the N-terminus of mouse IgG2a Fc fragment contained in an expression vector. The protein was expressed by transient transfection of 293F or 293T cells with the expression plasmid and purified by Protein A affinity chromatography. The biotinylated SIRPα-d1 protein was prepared using Sulfo-NHS-SS-Biotin (Thermo).

Preparation of Purified Phage-scFvs for ELISA or Neutralization Assay

The phage-scFvs in the supernatant of 10-30 mL bacterial culture were precipitated by PEG/NaCl and then quantified by a spectrometer. Activities of different phage-scFvs for antigen binding or SIRPα competition assays were evaluated based on the dose-response of serially diluted phage-Abs that was normalized to the same concentration.

Preparation of scFv-hFc Minibodies

ScFv encoding gene from the phage-scFv expressing vector was subcloned into an expression vector containing human IgG1 Fc fragment at C-terminus of the scFv. To produce scFv-hFc, 293F (Life Technologies) or 293T cells (ATCC) were transiently transfected with the scFv-hFc expression plasmid, 5 days after transfection, the cell culture supernatant were harvested and scFv-hFc was purified by Protein A affinity chromatography (Protein A Sepharose CL-4B, GE Healthcare).

Preparation of Full-Length IgG1 Antibody

The VH and VL coding sequence of a scFv were separately subcloned into antibody heavy chain (HC) expression vector and light chain (LC) expression vector. To make IgG1 antibody, 293F or 293T cells were transiently co-transfected with the two expression plasmids (HC+LC plasmids) at a 1:1 ratio. 5 days after transfection, the cell culture supernatant were harvested for purification of IgG1 by Protein A affinity chromatography.

ELISA Assay

CD47 binding ELISA: 2 μg/mL of neutravidin (Sigma) in phosphate buffered saline (PBS) was coated in U-bottom 96-well plate (Nunc, MaxiSorp™), 100 μL per well, at 4° C. overnight or 37° C. for 1 hour. 2 μg/mL of bio-CD47 in PBS containing 2% nonfat milk were then captured onto the plates at 100 μL per well by incubation at 30° C. for 1 hour. For phage-scFv based ELISA, serial diluted phage-scFvs in PBS containing 2% nonfat milk were added to each well at 100 μL per well. Specific bound phage-scFvs were detected by adding HRP-conjugated mouse anti-M13 antibody (GE Healthcare) and incubated for 30 min at 30° C. In between each incubation step, the ELISA plate was washed for 6 times with PBST solution (0.05% Tween20 containing PBS) at 200 μL per well. Followed by HRP-conjugated antibody incubation, the ELISA signal was developed by incubating with TMB substrate (Sigma) for 5-10 mins at 30° C. and then stop the reaction with 2M H₂SO₄ at 50 μL per well. The absorbance at 450 nm was read by a microplate reader (Bio-Rad). For scFv-Fc or IgG1 based ELISA, the method was basically the same as described above for phage-scFvs except the bound antibodies were detected by HRP-conjugated mouse anti-human IgG Fc antibody (Sigma).

SIRPα competition ELISA: 2 μg/mL of CD47-ECD-His₆ at 100 μL per well were coated onto U-bottom 96-well plate (Nunc, MaxiSorp™) at 4° C. overnight or 37° C. for 1 hour. 0.8 nM or 0.5 nM of bio-SIRPα-d1-mFc was mixed with different concentrations of testing antibodies or SIRPα-d1-mFc in PBS containing 2% nonfat milk, and then were captured onto the plates at 100 μL per well by incubation at 30° C. for 1 hour. Specific bound bio-SIRPα-d1-mFc were detected by adding HRP-conjugated streptavidin (Sigma) and incubated for 30 min at 30° C. The wash, development and read steps are the same as hCD47 binding ELISA. The OD450 values were transformed to Inhibition % in data analysis.

Surface Plasmon Resonance (SPR) Analysis

Surface plasmon resonance measurements were performed on a Biacore T200 instrument (BD) or Biacore X200 (BD). Anti-CD47 antibodies were captured onto an immobilized anti-human IgG Fc CM5 biosensor chip generated using standard primary amine coupling. All measurements were done in HBS-EP buffer with pH7.4 or pH6.8 or pH6.0 at 25° C., and magnesium chloride was used for surface regeneration. Serial dilutions of CD47 were injected over each flow cell at a flow rate of 30 μl/min. All data were analyzed with the Biacore T200 evaluation software with a 1:1 Langmuir binding model.

SPR was performed on a Biacore X200. 1 μg/mL of anti-CD47 scFv-hFcs were captured onto a CM5 chip for 30 s at 10 μl/min. CD47 in 2-fold serial dilutions (starting at 32 nmol, five concentrations total) were injected over the antibody-bound surface for 1 min at 30 μl/min followed by a 1 min dissociation phase. After each cycle, the surface was regenerated with magnesium chloride.

Generation of CHO-47 Stable Cell Line

cDNA of human CD47 (Form2, 305 amino acids) were cloned from total cDNA of human PBMC, and were introduced into expression plasmid containing neo resistance sequence. After transfected with the hCD47 expression plasmid, CHO cells that had been stably transfected with human CD47 (CHO-47) was obtained by selection with 1 mg/mL G418, and was sorted to equivalent surface expression by flow cytometry on a FACSAria (BD).

Flow cytometry Assay

For CD47 binding test, CHO-47, or other cell lines expressed hCD47 were incubated with serial diluted anti-CD47 antibodies at 4° C. for 1 hour. Then, the cells were incubated with FITC-labeled anti-human Fc gamma specific antibody (Sigma) at 4° C. for 0.5 hour. For SIRPα competition FACS, 10 nM of bio-SIRPα-d1-mFc was mixed with different concentrations of testing antibodies and incubated with CHO-47, or other CD47 positive cells at 4° C. for 1 hour. Then, the cells were incubated with FITC-labeled anti-Streptavidin specific antibody (Sigma) at 4° C. for 0.5 hour. For non-specific binding FACS, cells that do not expressed hCD47, such as CHO, were incubated with high concentration, such as 500 nM, of testing antibodies. The same as CD47 binding test, cells then were incubated with FITC-labeled anti-human Fc gamma specific antibody (Sigma). After incubation, the cells were washed for 3 times with PBS solution containing 0.5% BSA. Then cells were resuspended in 400 μL of PBS without BSA, antibodies bound to cells were detected by flow cytometry on a LSR II (BD).

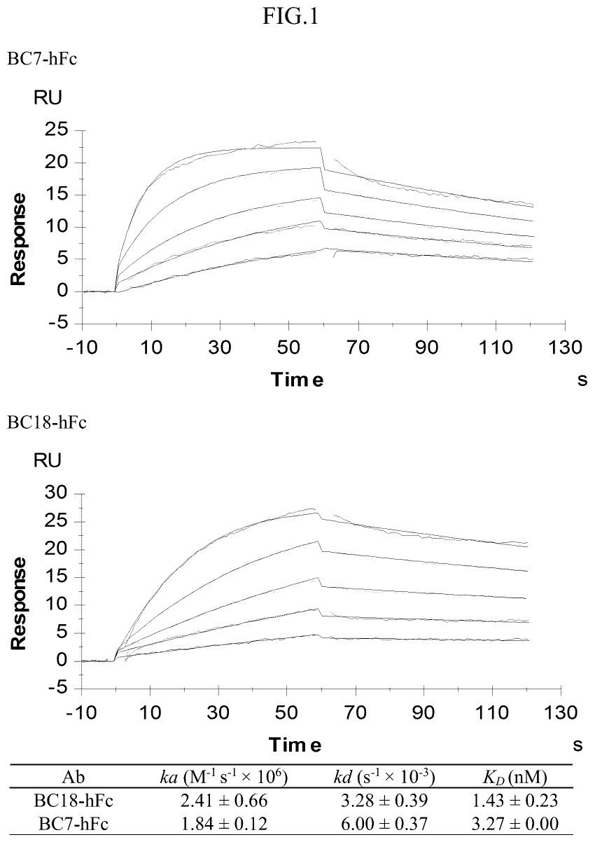

Through the above described ELISA, SPR analysis, we identified two top antibodies, BC18 and BC7. In SPR assay, BC18 showed the best kinetic performance with a KD value of 1.43 nM; BC7 followed with a KD value of 3.27 nM (FIG. 1 and table 2). Thus, BC18 and BC7 were chosen for further identification.

Further Characterization of BC18 and BC7 In Vitro

Comparison of BC18 and BC7 with Other Published Therapeutic CD47 mAbs.

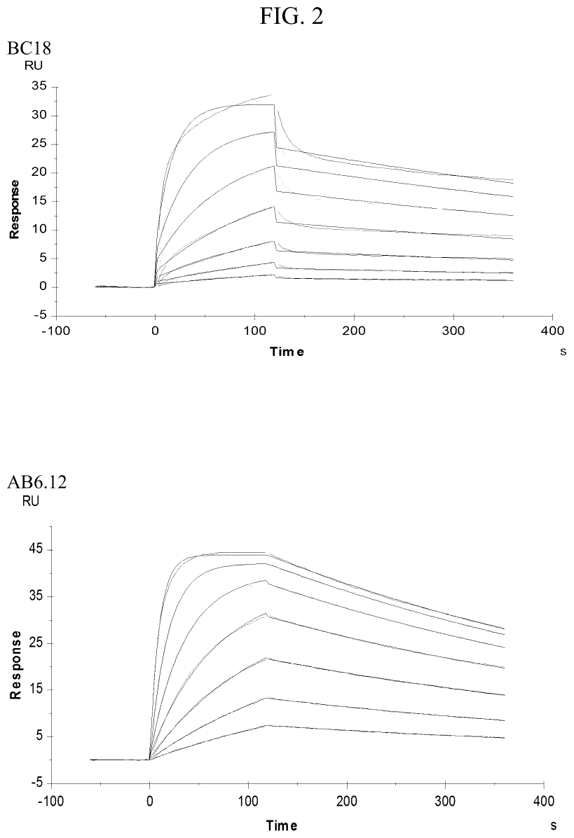

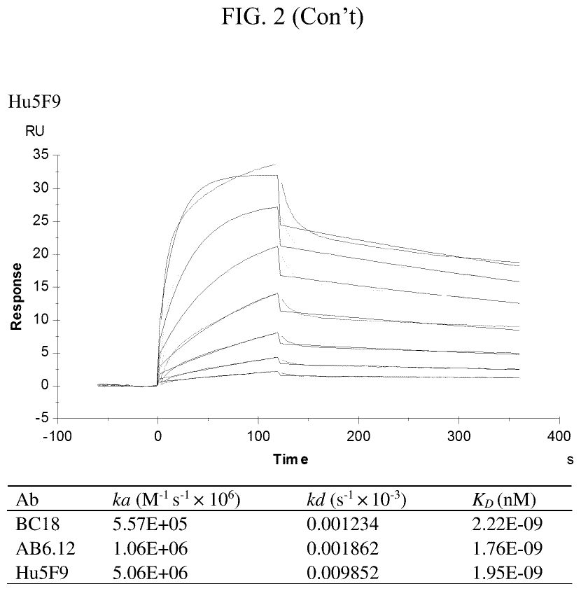

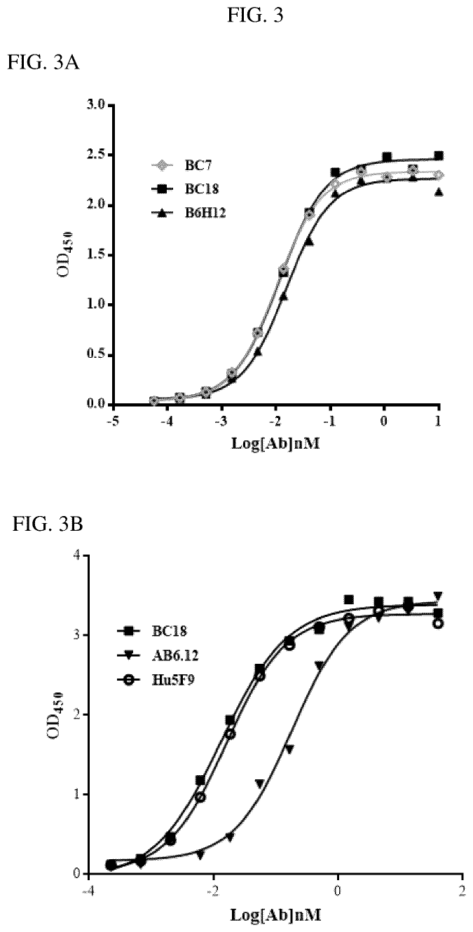

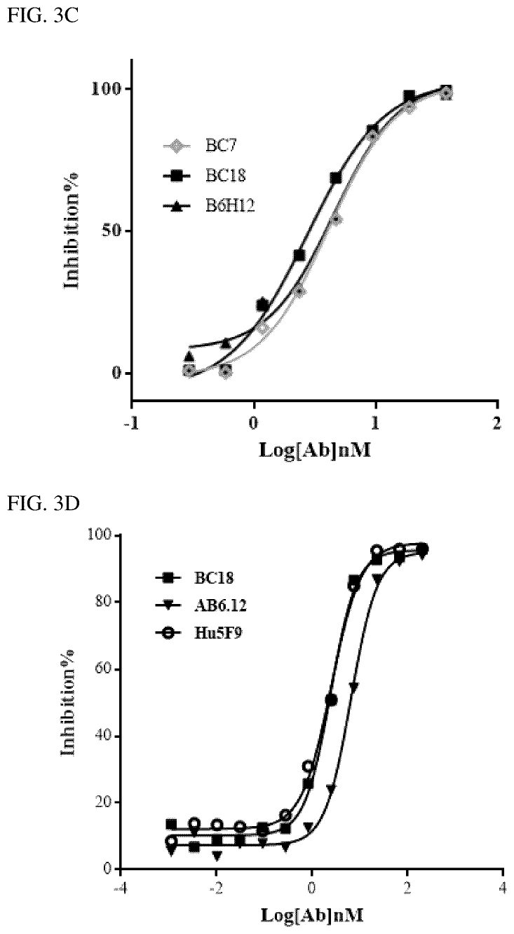

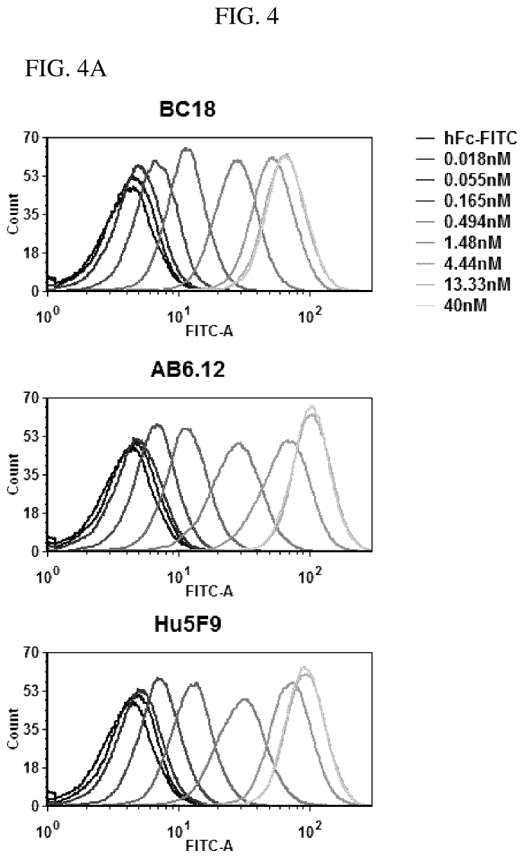

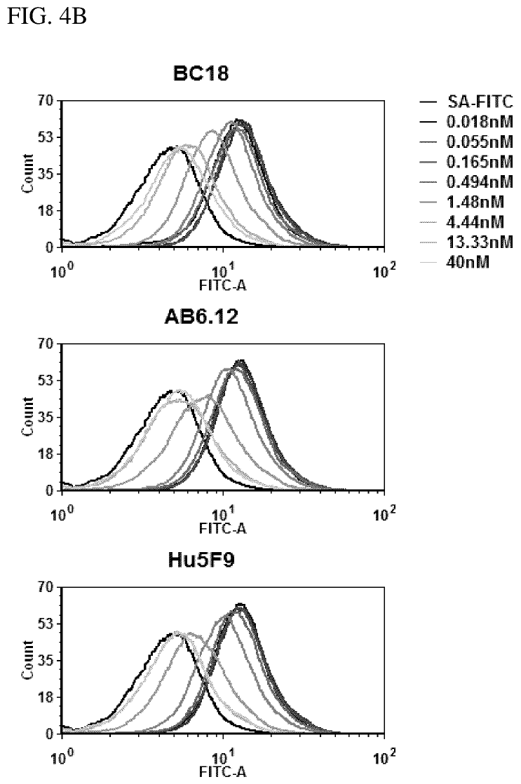

BC18 and BC7 were next converted to full-length human IgG1 form. Then, the CD47 binding affinity and SIRPα competition ability were compared with B6H12, AB6.12, Hu5F9 by SPR assay (FIG. 2), ELISA (FIG. 3) and FACS (FIG. 4). B6H12 is a published CD47 antibody showing broad-spectrum tumor therapeutic effect. AB6.12 and Hu5F9 are two anti-CD47 mAbs under phase I clinical trial(WO2011/143624A2; US2013/0224188A1). BC18 showed different kinetic performance with comparable KD value and to AB6.12, Hu5F9. In the ELISA assay, both BC18 and BC7 showed stronger CD47 binding and SIRPα competition activities than B6H12 (FIGS. 3A and C) BC18 was better than AB6.12 and similar to Hu5F9 in both CD47-binding and SIRPα competition activities (FIGS. 3B and D). At cell surface level, BC18 showed a comparable CD47 binding and SIRPα competing activities with AB6.12 and Hu5F9 (FIG. 4).

In FIG. 2, SPR was performed on a Biacore T200. 1 μg/mL of anti-CD47 full length IgG1 antibodies were captured onto a CM5 chip for 30 s at 10 μl/min. CD47 in 2-fold serial dilutions (starting at 100 nmol, seven concentrations total) were injected over the antibody-bound surface for 120 s at 30 μl/min followed by a 240 s dissociation phase. After each cycle, the surface was regenerated with magnesium chloride.

FIG. 3 shows comparison of the anti-hCD47 antibodies on the CD47 binding and SIRPα competition abilities by ELISA. In FIG. 3, A. Antibodies in the form of full-length IgG1 were 3-fold serial diluted (starting at 10 nM, 12 concentrations total). B. Antibodies in the form of full-length IgG1 were 3-fold serial diluted (starting at 40 nM, 12 concentrations total). C-D. SIRPα competition ELISA. C. 0.8 nM of biotinylated SIRPα-d1-mFc were mixed with tested full-length IgG1 or SIRPα-d1-mFc in 2-fold serial dilutions concentration (starting at 40 nmol, 8 concentrations total). D. 0.5 nM of biotinylated SIRPα-d1-mFc were mixed with tested full length IgG or SIRPα-d1-mFc in 3-fold serial dilutions concentration (starting at 200 nmol, twelve concentrations total).

FIG. 4 shows comparison of the anti-hCD47 by FACS analysis. In FIG. 4, A. CD47 Binding FACS. Antibodies in the form of full-length IgG1 were 3-fold serial diluted (starting at 40 nmol, eight concentrations total). B. SIRPα competition FACS. 100 nM of biotinylated SIRPα-d1-mFc were mixed with tested full-length IgGs in 3-fold serial dilutions concentration (starting at 40 nmol, eight concentrations total). SA-FITC represents FITC labeled streptavidin.

BC18 and BC7 Bound to the CD47 Expressed on Tumor Cells

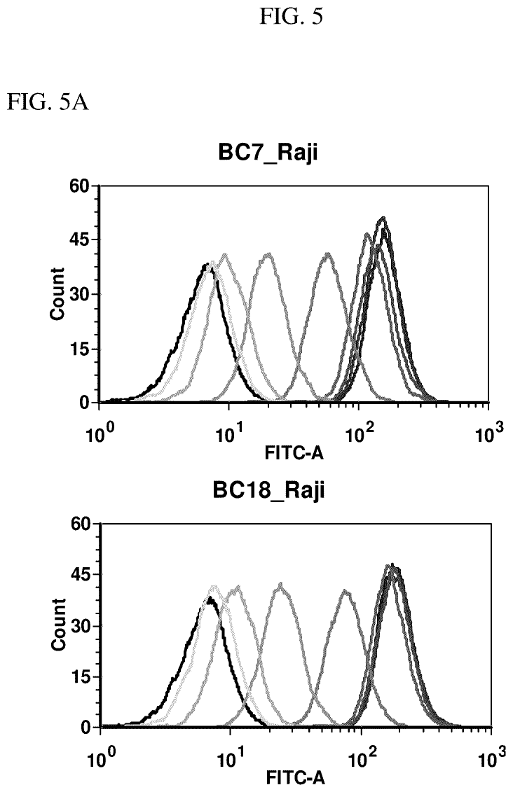

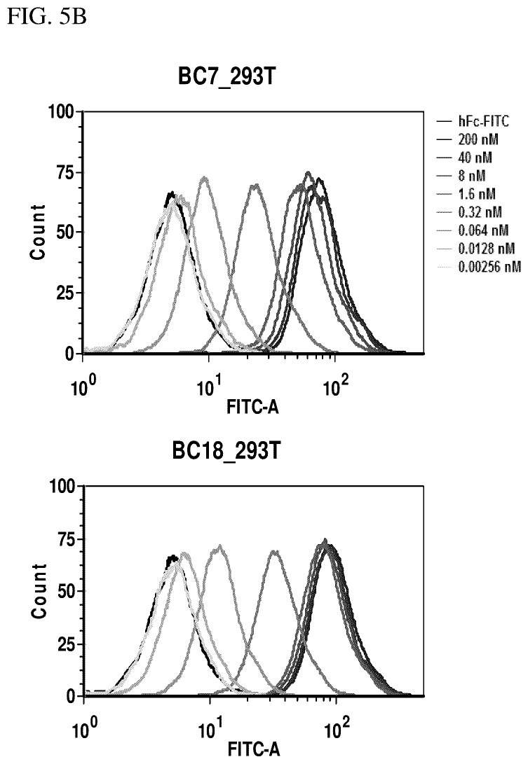

Two human cell lines expressed human CD47, Raji and HEK-293 were used in this experiment. Raji is a human B lymphoblast cell line; HEK-293T is a human embryonic kidney cell line. Binding of BC18 was detected with FITC anti-human Fc antibody and analyzed by BD LSR II. (FIG. 5) BC18 and BC7 can bind to hCD47 on cell surface of Raji and HEK-293 even at low antibody concentration of 0.06 nM. Cell surface hCD47 binding affinity of BC18 is slightly stronger than BC7.

In FIG. 5, it is shown that BC18 and BC7 can bind to cell surface CD47. Raji (A) or 293T (B) cells were incubated with 5-fold serial diluted (starting at 200 nmol, eight concentrations total) antibodies in the form of full length IgG1. After incubated with FITC conjugated anti-hFc, cells were analyzed by BD LSR II.

In Vitro Phagocytosis Assay

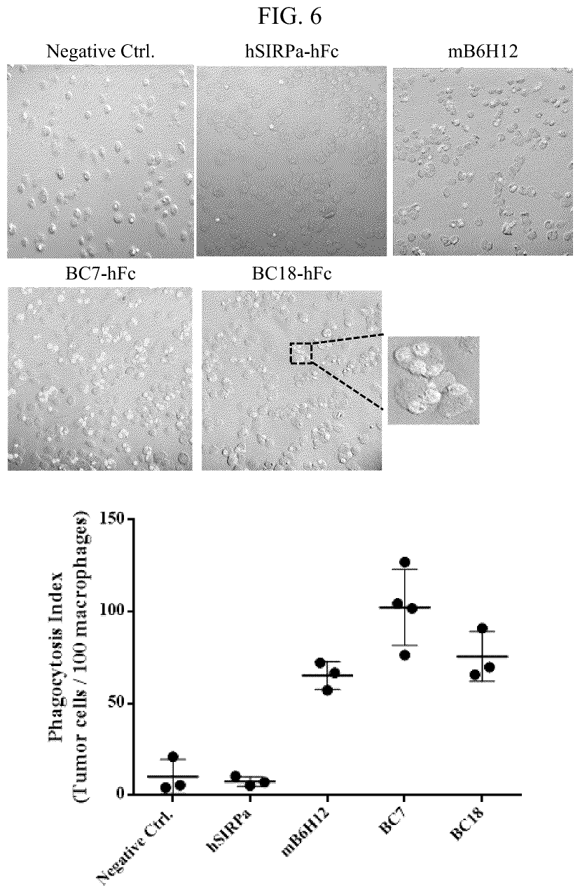

We then investigated whether BC18 and BC7 blockade of the CD47/SIRPα interaction enabled phagocytosis of target cells. The HL-60 cell line was used as the target cells and labeled with CF SE. Phagocytic activity was measured by using human peripheral blood-derived macrophages and counting the number of ingested cells using fluorescence microscopy. Both BC18 and BC7 induced potent phagocytosis of HL-60 cells, and acted better than hSIRPα-hFc and mB6H12. (FIG. 6)

In Vitro Phagocytosis Assay

Human macrophages were derived from peripheral blood mononuclear cells (PBMC). Human B lymphoblast cell (Raji), HL-60 or Jurkat was selected as targeted cells. Targeted cells were labeled with 5 μM carboxyfluorescein succinimidyl ester (CFSE). Anti-CD47 antibodies with hFc were added into CFSE+ Raji cells at a concentration of 10 μg/ml. After 2 h incubation, opsonized targeted cells were co-cultured with macrophages maintained with RPMI Medium 1640 at 37° C. Pictures of 3 distinct points of every sample were taken by confocal, at least 200 macrophages were counted to calculate how many targeted cells are engulfed by 100 macrophages.

FIG. 6 shows BC18 and BC7 induced potent macrophage-mediated phagocytosis of HL-60. In FIG. 6, HL-60 was labeled with CFSE and incubated with human peripheral blood-derived macrophages in the presence of 10 ug/ml BC18, BC7 in form of scFv-hFc, hSIRPα-hFc, mB6H12. PBS was used as negative control. Two hours later, macrophages were imaged by confocal to determine the phagocytic index (number of target cells ingested per 100 macrophages) in duplicate. Lines indicate mean values.

BC18 can Reduce and Even Cure Human Lymphoma or Breast Cancer.

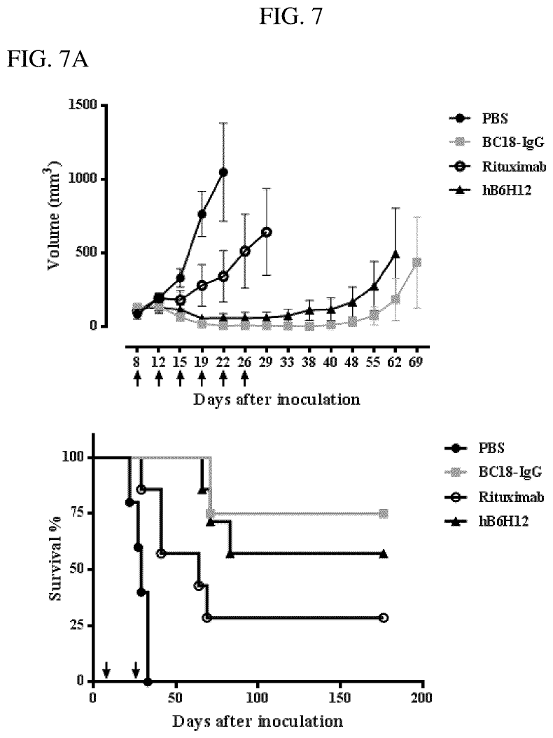

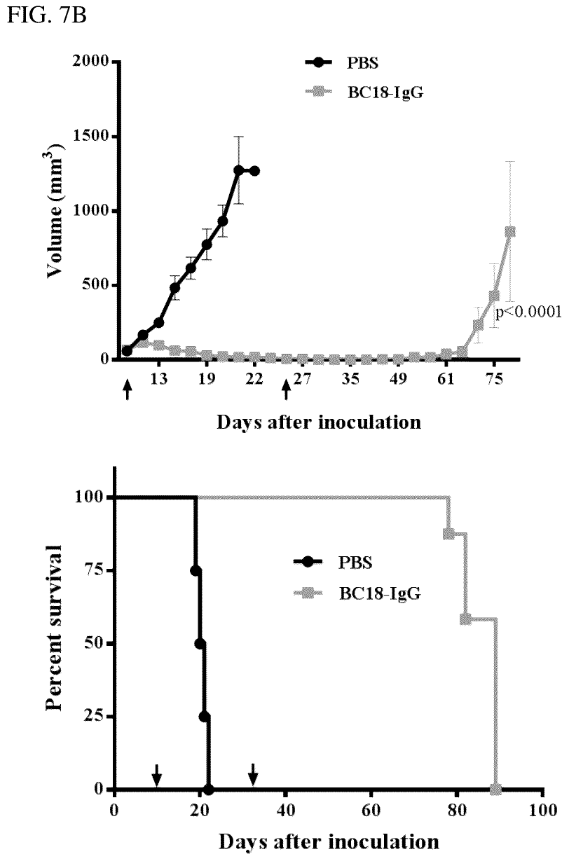

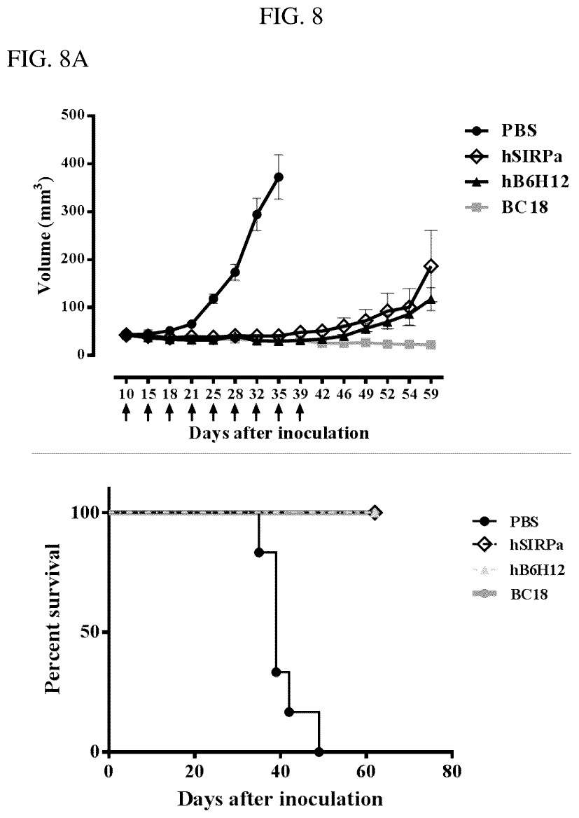

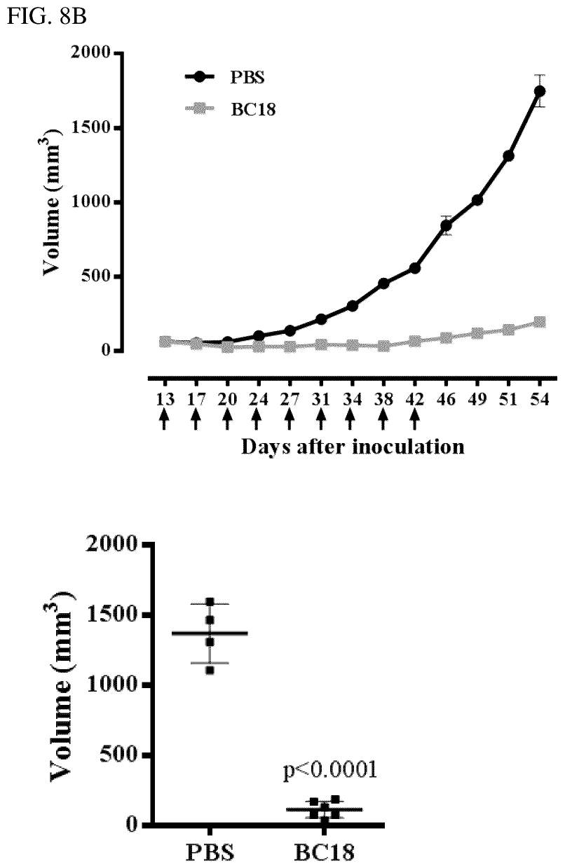

We next investigate the ability of BC18 to eliminate human cancer cells in vivo. Raji, a lymphoma cell line, was engrafted on right frank of NOD-SCID (FIG. 7A) or NSG (FIG. 7B) mice by subcutaneous injection. Vehicle (PBS), BC18, or together with hB6H12, hSIRPα-hFc at 10 mg/kg were administered biweekly by intraperitoneal for six time. Analysis of the tumor size during the experiment showed a significant reduction in tumor burden and increase of survival in mice treated with BC18. Meanwhile, BC18 was more efficient than hB6H12 and hSIRPα in NOD-SCID group. MDA-MB-231, a breast adenocarcinoma cell line, was also used to engraft NOD-SCID (FIG. 8A) or NSG (FIG. 8B) mice on mammary fat pad by subcutaneous injection. When palpable tumors formed, these mice were also administered PBS, BC18 or together with hB6H12, hSIRPα at 10 mg/kg twice a week for three weeks. BC18 also showed therapeutic effect against MDA-MB-231.

FIG. 7. shows that BC18 can significantly reduce tumor size of human lymphoma in immunodeficient mice. 1×10⁶ Raji cells were engrafted subcutaneously on the right lower flank of NOD-SCID mice (A) or NSG mice (B). Left). Tumor volumes. Right). Survival curve. Tumor dimensions were measured to calculate volumes according to the ellipsoid formula (π/6×length×width²). Antibodies used were in form of full length IgG1. Curves indicate mean values of each groups. Each black arrow represents one injection or the beginning and the end of treatment. Every mouse was injected for 6 times.

FIG. 8. shows that BC18 can significantly reduce tumor size of human breast cancer in immunodeficient mice. 1×10⁶ MDA-MB-231 cells were engrafted subcutaneously on mammary fat pad of NOD-SCID mice (A) or NSG mice (B). A. Left). Tumor volumes. Right). Survival curve. B. Left). Curve of tumor volumes. Right). Tumor volumes measured in vitro on day 54 post inoculation. Tumor dimensions were measured to calculate volumes according to the ellipsoid formula (π/6×length×width²). Antibodies used were in form of full length IgG1. Curves indicate mean values of each groups. Each black arrow represents one injection or the beginning and the end of treatment. Every mouse was injected for 6 times.

Further Modification of BC18 and BC7.

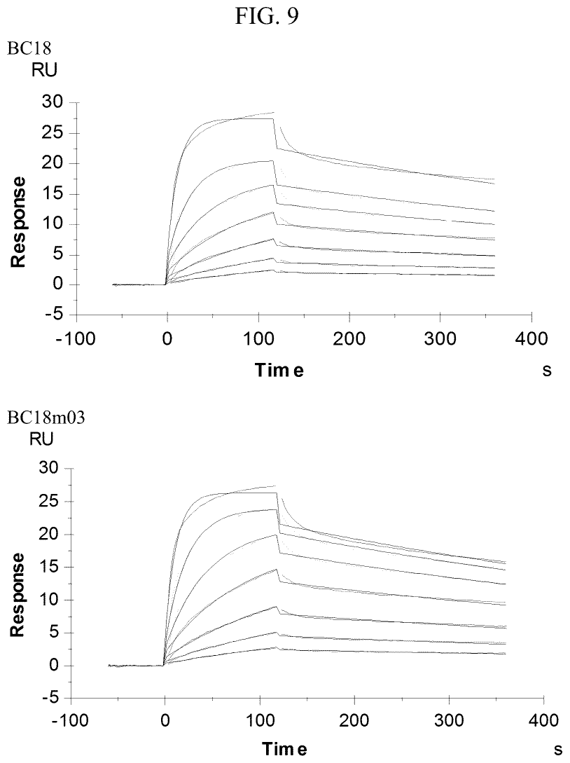

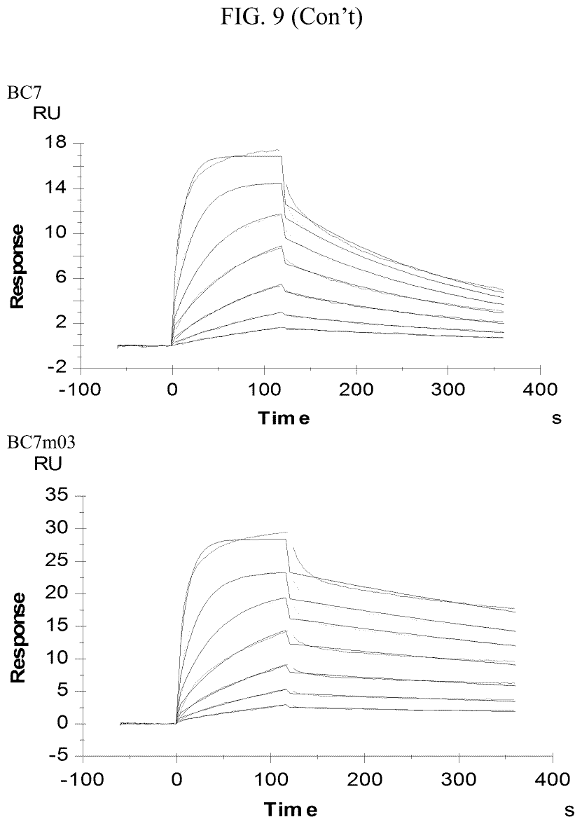

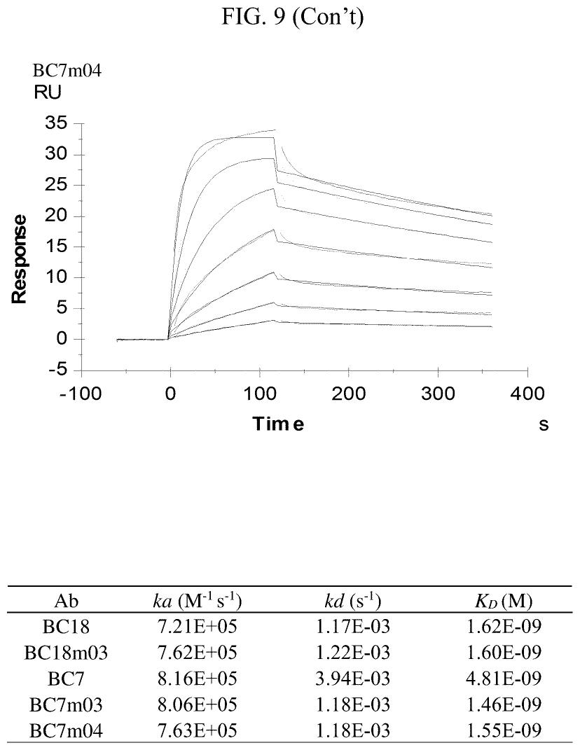

The sequences of BC18 and BC7 are very similar. For reducing immunogenicity and improve stability, BC18 and BC7 were mutated to BC7m03, BC7m04, BC18m03 according to their germline sequence (IGKV1-12.01, IGKV1D-16.01, IGHV4-59.08). These mutants have similar kinetic performance as BC18 and better than BC7 (FIG. 9). At the same time, their thermal stability is improved (Table 2). Moreover BC18, BC18m03, BC7m03 and BC7m04 shows no binding activity to CHO, Hepa1-6, B16F10, BHK-21, CT26 cell lines that do not expressed human CD47 even at high antibody concentration of 500 nM.

FIG. 9. shows binding kinetics of BC18, BC7 and their mutants by Surface Plasmon Resonance (SPR) analysis. SPR was performed on a Biacore T200. 1 μg/mL of anti-CD47 full length IgG1 antibodies were captured onto a CM5 chip for 30 s at 10 μl/min. CD47 in 2-fold serial dilutions (starting at 100 nmol, seven concentrations total) were injected over the antibody-bound surface for 120 s at 30 μl/min followed by a 240 s dissociation phase. After each cycle, the surface was regenerated with magnesium chloride.

Thermal Stability Assay

Thermal stability of antibodies was tested by Protein Thermal Shift Dye Kit (Thermo Fisher) under the user guide. In brief, 12.5 μL antibody at 0.5 mg/mL or PBS was mixed with 5 μL Protein Thermal Shift buffer and 2.5 μL Diluted Protein Thermal Shift Dye, and added into reaction plate pre-cooled on ice. Thereafter, load the plate on 7500 Fast Real-Time PCR System, run melt curve experiment. Tm values were analyzed by Protein Thermal Shift software.

TABLE 2
TmD values of BC7, BC18 and their mutants
tested by Protein Thermal Shift Dye Kit

| Protein | TmD value |
|---|---|
| BC18 | 78.42 ± 0.12 |
| BC18m03 | 80.66 ± 0.14 |
| BC7 | 84 ± 0.12 |
| BC7m03 | 85.17 ± 0.5 |
| BC7m04 | 85.6 ± 0.2 |

### Example 2 Bispecific Antibody

Materials & Methods

Cell Lines

Free Style 293F cells were cultured following manufacturer instructions (Thermo Fisher Scientific). Raji cells were from the Cell Bank of Type Culture Collection, Chinese Academy of Sciences. The Raji-GPC3^{H} cell line (expressing human GPC3) was generated via electroporation followed by G418 selection and single cell clone isolation. The human HCC cell line Hep3B was generously provided by Dr. Fengmin Lu (Peking University Health Science Center, Beijing, China). The Hep3B-Luc23 cell line was constructed as previously described. The CD47 gene knockout cell lines Hep3B-CD47^{KO} and Jurkat-CD16A-CD47^{KO} were generated with CRISPR-Cas9 technology. Raji, Raji-GPC3^{H}, and Jurkat-CD16A-CD47^{KO} cells were cultured with RPMI 1640 medium supplemented with 10% fetal bovine serum (FBS). Hep3B, Hep3B-Luc23, and Hep3B-CD47^{KO} cells were cultured with Dulbecco's Modification of Eagle's Medium (DMEM) supplemented with 10% FBS.

Bispecific Antibody Construction

The variable heavy chain (VH) and light chain (VL) gene sequences of GC33 were synthesized by GenScript. Anti-human CD47 antibody BC18 was selected from our human non-immune scFv (single-chain fragment of variable domain) phage display antibody library. And the genes of its VH and VL were sequenced. The coding sequences of the VH and VL of GC33 and BC18 were subcloned, respectively, into a human IgG1 heavy chain (HC) expression vector and a light chain (LC) expression vector.

To construct bispecific antibodies, we modified the original wide-type HC and LC expression vectors (blank) using previously reported KiHs and CrossMab technologies. Briefly, several mutations were introduced into the CH3 domain of the HC via site-directed mutagenesis to generate the “knob chain” (T366W, S354C) and the “hole chain” (Y349C, T366S, L368A, Y407V). The CH1 domain of the hole chain and the CL domain of the LC were interchanged to respectively generate the paired crossover heavy chain (CL-hole chain) and light chain (CH1-LC). The VH and VL fragments of GC33, BC18, and a control antibody were then cloned into the knob chain, CL-hole chain, or CH1-LC expression vectors as needed.

Surface Plasmon Resonance (SPR) Analysis

All SPR analyses were performed on a Biacore T200 instrument (Biacore, GE Healthcare). To measure the binding affinity of each monoclonal antibody to their own antigen, GC33 or BC18 (isotype human IgG1) were first captured using a protein A/G (Pierce, Thermo Fisher) immobilized to a CM5 sensor chip; the analytes (hGPC3-ΔHS or hCD47 proteins, see Supplemental Materials and Methods) were then injected over each flow cell at serially diluted concentrations. The association rates (Kₒₙ), dissociation rates (K_{off}), and affinity constants (KD) were calculated using BiacoreT200 evaluation software. Kinetics analyses of GPC3/CD47 biAb or GPC3/CD47-DANG variant binding to mFcγRs were performed as described above (here the analytes were serial diluted mFcγRs).

To demonstrate simultaneous binding of GPC3/CD47 biAb, hGPC3-ΔHS was covalently attached to a CM5 sensor chip using an amine coupling kit (Biacore) at a surface density of ˜600 response units (RU). GPC3/CD47 biAb was injected at 20 μg/mL with a dissociation phase of 80 s, and 1 mM hCD47-mouse Fc (mFc) fusion protein was then injected with a dissociation phase of 120 s followed by regeneration with 3 M MgCl₂.

FACS-Based Binding and Blocking Assays

To compare binding abilities of each antibody to single antigen or dual antigen-expressing cells, Raji or Raji-GPC3^{H} cells (5×10⁵ per well in 96-well plates) were incubated with 0.4 μg/mL or 2 μg/mL of test antibodies for 30 min at 4° C. in FACS buffer (0.5% BSA/PBS). Antibody bound cells were then washed and incubated with FITC-conjugated goat anti-human IgG secondary antibody (Sigma-Aldrich) for 20 min at 4° C. These samples were analyzed with a FACS Arial II instrument (BD Biosciences) and data was processed using FCS Express ver.4 (De Novo Software).

To assess the binding selectivity of these antibodies, Raji cells were labeled with CellTrack™ Deep Red according to the manufacturer's protocol (Thermo Fisher Scientific), followed by mixing with unstained Raji-GPC3^{H} cells at a 1:1 ratio. The mixed cells (1×10⁶ per well) were then incubated with 0.2 μg/mL of each antibody for 30 min at 4° C. and analyzed as described above.

To assess the abilities of all anti-CD47 antibodies to block the interaction between CD47 and SIRPα, Raji or Raji-GPC3^{H} cells were incubated with 50 nM of biotinylated SIRPα-mFc in the presence of different anti-CD47 antibodies (10 μg/mL) at 4° C. for 30 min. Streptavidin-FITC (Sigma-Aldrich) was then added after washing to measure the binding of SIRPα to each type of cells.

Red Blood Cell Agglutination Assay

Human whole blood collected from healthy donors was washed and resuspended with PBS. 3 million RBCs in PBS were plated per well in polypropylene 96-well plates (Corning) and incubated with serially diluted antibodies for 1 hr at room temperature.

Generation of hCD47/hSIPRα Humanized Mice

All animal experiments were conducted following the National Guidelines for Housing and Care of Laboratory Animals in China and performed under the approved IACUC protocols at National Institute of Biological Sciences, Beijing. The humanized mice were generated by CRISPR/Cas9-mediated gene editing technology. The humanized Sirpα mice were developed by replacing the exon 2 of mouse Sirpα gene that encodes the extracellular domain with human Sirpα exon counterpart. Similarly, the humanized Cd47 mice were developed by replacing the exon 2 of mouse Cd47 gene that encodes the extracellular domain with human Cd47 exon counterpart. The hCd47/hSirpα double gene-humanized mice were developed by mating the two types of humanized mice together. The homozygous humanized mice were used in this study.

Pharmacokinetics and Hematologic Toxicity Studies in Mice

A single-dose pharmacokinetics (PK) study was carried out in hCD47/hSIRPα humanized mice. 8- to 10-week-old humanized mice (n=3 per group) were i.p. injected with 10 mg/kg of each antibody. Blood samples were collected at different time points, and serum concentrations of each human antibody were measured with a human IgG ELISA quantitation kit (Bethyl Laboratories). The evaluation of the PK data was conducted with WinNonlin software. 6- to 8-week-old humanized mice (n=4-6 per group) were i.p. injected with 10 mg/kg of each antibody. Blood samples were collected from orbital sinus 45 min or 2.5 hrs after antibody administration and the red blood cell counts were measured using ADVIA 2120i instrument at Beijing BrightShines Ltd.

ADCC Reporter Bioassay

The ADCC reporter gene bioassay was performed according to a previous report. Target cells (Raji, Raji-GPC3^{H} or Hep3B) were seeded at 1.5×10⁴ into each well of a 96-well solid white polystyrene microplate (Corning). Three fold serially diluted anti-GPC3 mAb, anti-CD47 mAb, or GPC3/CD47 biAb, as well as Jurkat-CD16A-CD47^{KO} effector cells were added; the final effector to target cell ratio (E:T) was 6:1. Luciferase activity was measured using a luminescent substrate (Promega Bright-Glo™) after incubation for approximately 8 hrs at 37° C., 5% CO₂.

ADCP Assays

Mouse bone marrow derived macrophages (BMDMs) were used as effector cells in ADCP assays. To prepare BMDMs, mouse bone marrow cells were collected from the femurs and tibia of hCD47/hSIRPα humanized mice and induced by DMEM medium supplemented with 15% L929 (secreting GM-CSF) cell culture medium for 3 days. The differentiated BMDMs were labeled with a 1:200 dilution of anti-mouse F4/80-Alex Fluor647 (Thermo Fisher, clone BM8) prior to incubation with target cells.

For the competitive ADCP experiment, Raji and Raji-GPC3^{H} cells were used as the target cells; these were stained with CellTrace™ Yellow or CF SE according to the manufacturer's protocol (Thermo Fisher Scientific). The fluorescently labeled target cells were mixed at 1:1 and plated at a density of 4×10⁵ cells/well. For the HCC cell line ADCP experiment, Hep3B or Hep3B-CD47^{KO} cells were stained with CF SE and plated at a density of 8×10⁴ cells/well. Each target cell was incubated with 4 μg/mL of each antibody at RT for 10 min before being added to the differentiated and labeled BMDMs (˜2×10⁵ cells/well) at 37° C. for 2 hrs. Phagocytosis of fluorescent-labeled target cells by Alex Fluor647-labeled BMDMs was recorded using a Nikon A1R Confocal Microscope. Prior to microscopy imaging, un-phagocytosed cells were washed. Statistical analyses (multiple Student's t-tests or one-way ANOVA) were implemented in GraphPad Prism.

In Vivo HCC Xenograft Model

5×10⁶ Hep3B-Luc23 cells were injected subcutaneously into the right flank of 6- to 8-week-old, non-obese diabetic severe combined immunodeficiency (NOD-SCID) mice. Mice were randomized into groups (n=5 per group) based on equivalent mean tumor volumes and received 10 mg/kg of each antibody (or its variants) via i.p. injection twice a week for 3 weeks. In vivo tumor bioluminescence intensities of tumor-bearing mice were measured using an IVIS Lumina III In Vivo Imaging System (PerkinElmer) after i.p. injection of 15 mg/kg D-luciferin (PerkinElmer). Tumor dimensions were measured with an electronic caliper and tumor volume was calculated using the formula (L×W²)/2, where L and W are the largest and smallest measured diameters respectively. All mice were euthanized by CO₂ if their tumor size exceeded 2000 mm3 or at the end of the study.

For in vivo near-infrared fluorescence (NIRF) imaging, GPC3/CD47 biAb and an isotype control antibody were conjugated with Cy7 NHS ester (GE Healthcare). Hep3B tumor-bearing mice were randomized into two groups (n=3 per group) based on equivalent mean tumor volumes and injected with 8 mg/kg of each fluorescently labeled antibody. Mice were imaged 12, 24, 36, and 48 h after antibody administration, with excitation and emission wavelengths of 745 and 800 nm. Organs were dissected and imaged 48 h after antibody administration.

For depletion of macrophages, NOD-SCID mice were i.p. injected with 200 μL clodronate liposome (FormuMax) three days prior to initiation of GPC3/CD47 biAb treatment, followed by additional injection (100 μL) once a week until the end of the study. For depletion of neutrophils, NOD-SCID mice were i.p. injected with 200 μg anti-Ly6G antibody (1A8 clone, BioXCell) every five days.

Statistical Analysis

Statistical significance between different experimental groups was analyzed by Student's t-test, one-way ANOVA, or two-way ANOVA with Tukey's test; (*P<0.05, **P<0.01, ***P<0.001, ****P<0.0001). All statistical analyses and graph preparation were performed using GraphPad Prism.

### Example 2 Bispecific Antibodies

Generation of Bispecific Antibodies and Demonstration of GPC3/CD47 biAb's Greatly Improved Specificity for Dual Antigen-Expressing Cells

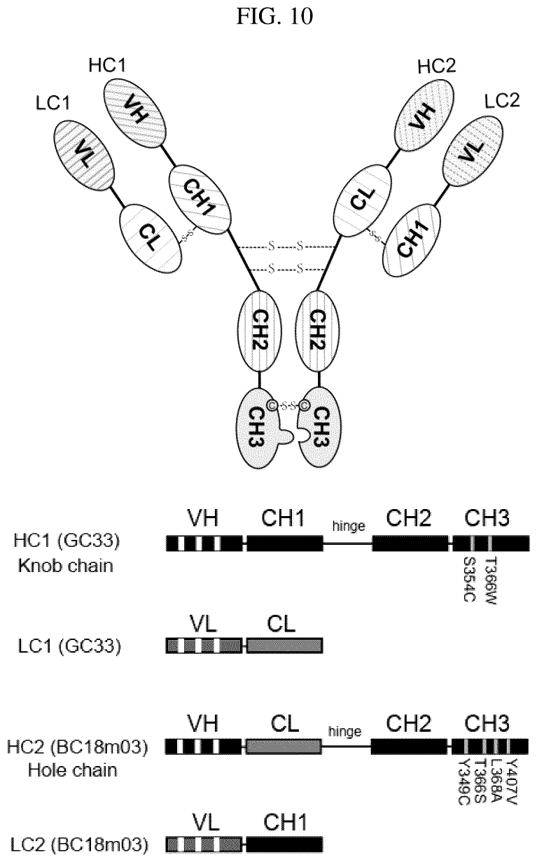

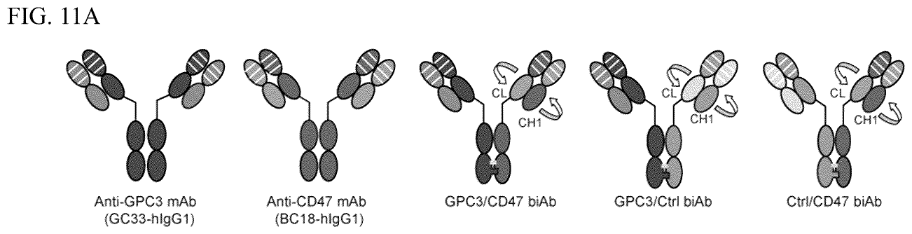

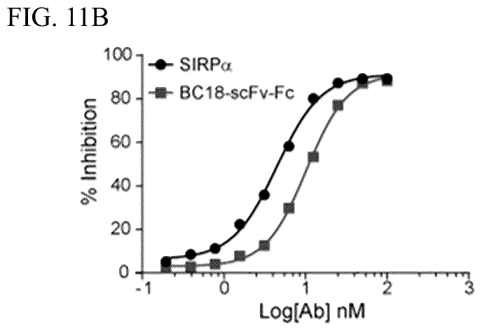

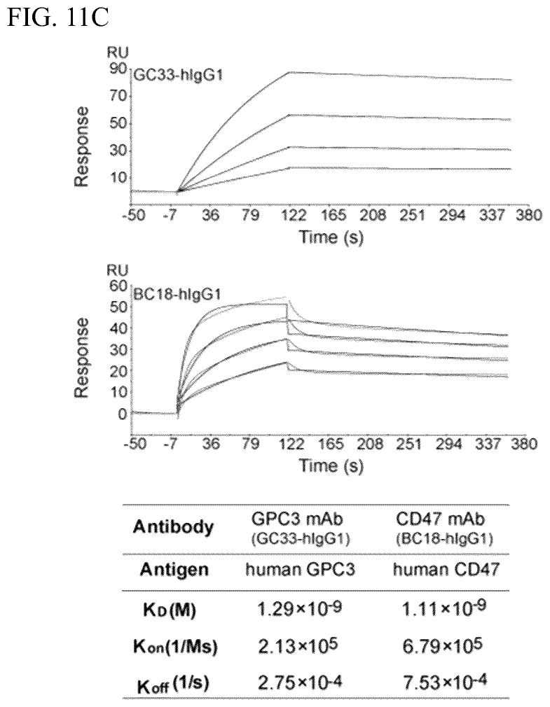

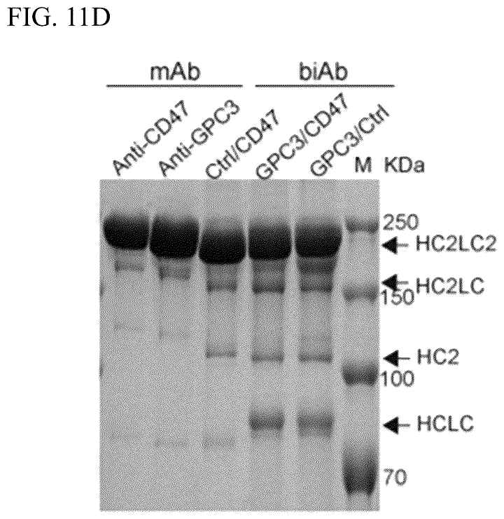

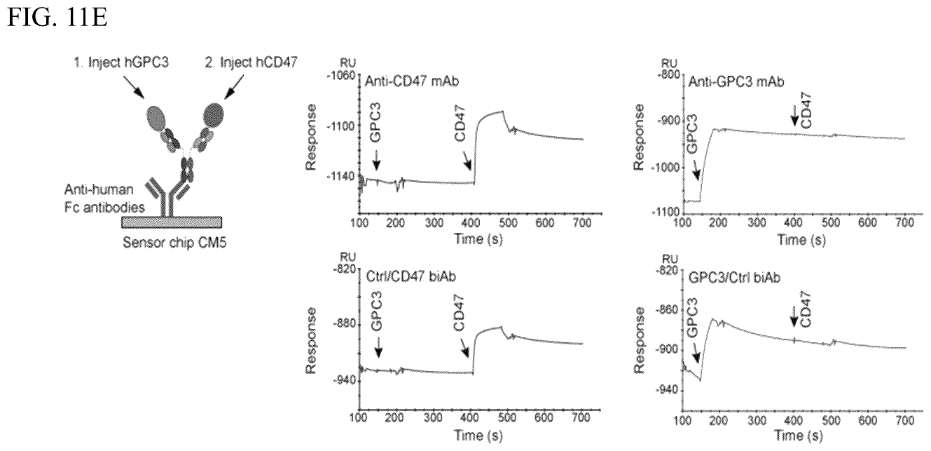

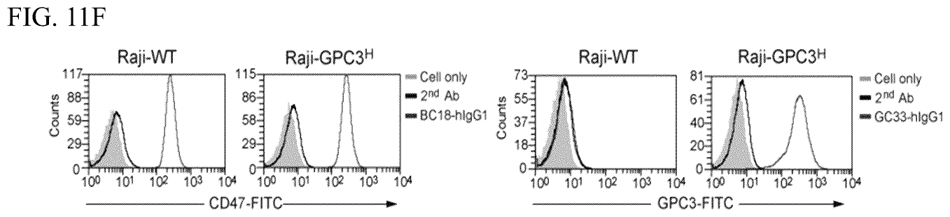

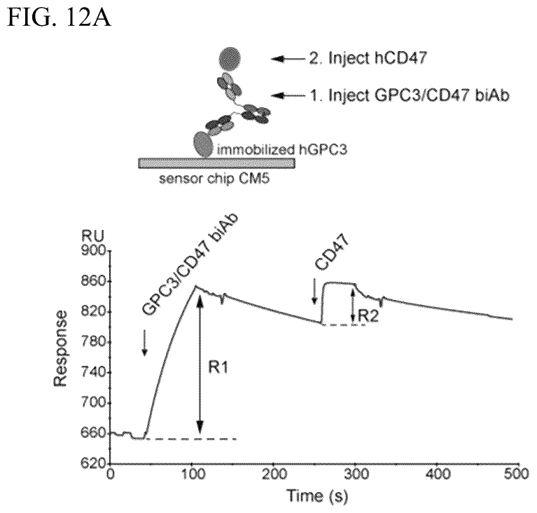

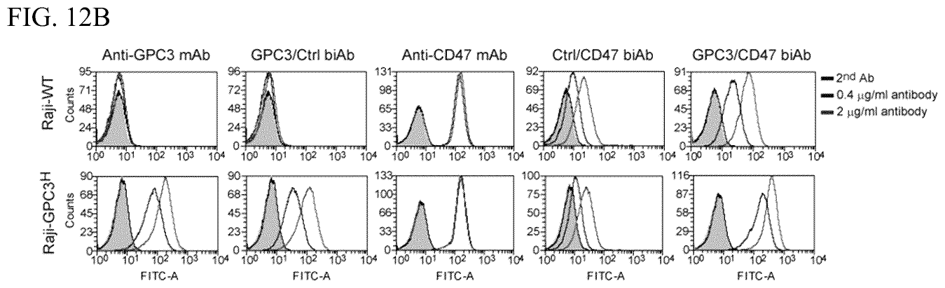

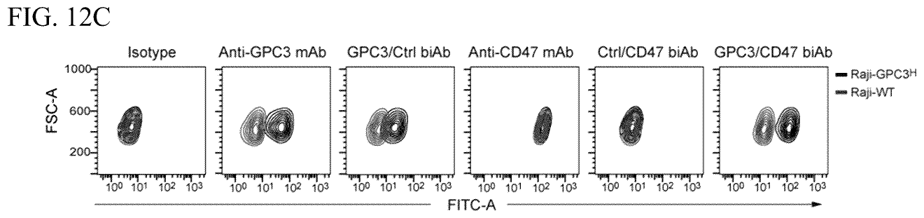

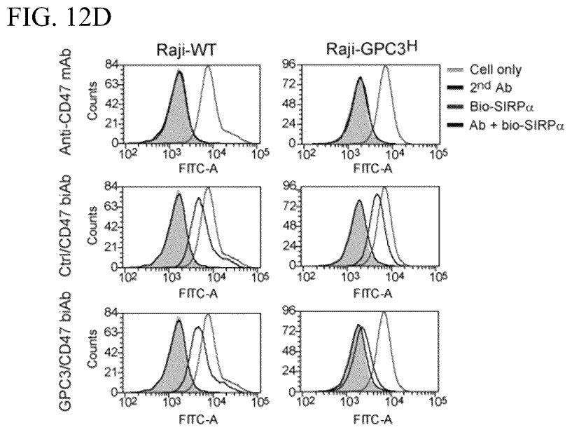

After characterizing the binding affinity, epitopes, and antitumor activities of a variety of anti-GPC3 or anti-CD47 mAbs, we adopted two antibodies—the human GPC3-targeting Codrituzumab (GC33) and the human CD47-targeting mAb (BC18)—to generate GPC3/CD47 biAb (FIG. 10 and FIG. 11A). BC18, obtained through screening of our phage display naïve antibody library (scFv), effectively blocks the interaction between CD47 and its receptor SIRPα (FIG. 11B). Both GC33 and BC18 have nanomolar range affinities for their own antigens as measured by surface plasmon resonance (SPR) analysis (FIG. 11C). Another two BsAbs (Ctrl/CD47 biAb or GPC3/Ctrl biAb) respectively for CD47 or GPC3 were generated as controls (FIG. 11A). All biAbs were produced via transient transfection of HEK293F cells, and were then purified via protein A affinity chromatography (FIG. 11D). Subsequently, SPR-based analysis demonstrated the dual specificity of the purified and properly assembled GPC3/CD47 biAb (FIG. 12A) and confirmed the respective specificities of the two control biAbs (FIG. 11E).

The widespread expression of CD47 on normal tissue cells may result in decreased antibody bioavailability to tumor cells, thus jeopardize the treatment efficacy of anti-CD47 antibodies (the so-called “antigen sink” phenomenon). We used an engineered GPC3+CD47+ double positive cells (Raji-GPC3^{H}, FIG. 11F) to examine if GPC3/CD47 biAb possesses selective binding properties for dual antigen-expressing cells as designed. Supporting that our dual-targeting strategy confers improved specificity, we found that GPC3/CD47 biAb had much stronger binding for double positive Raji-GPC3^{H} cells than for single antigen-expressing Raji-WT cells (FIG. 12B). Furthermore, we designed an in vitro experiment to mimic an antigen sink scenario where both double positive and single positive cells exist. Specifically, we labeled Raji cells with deep red dye and mixed them with Raji-GPC3^{H} cells at a 1:1 ratio prior to incubation with each antibody. During FACS analysis, cells were initially gated according to cell type, followed by analysis of antibody binding. GPC3/CD47 biAb exerts preferential binding for Raji-GPC3^{H} over Raji cells; no such specificity was evident for the aforementioned control anti-CD47 mAb or the Ctrl/CD47 biAb (FIG. 12C).

We next tested the SIRPα/CD47 blockade activity of GPC3/CD47 biAb: competitive FACS analysis showed that compared to anti-CD47 mAb and Ctrl/CD47 biAb—which each blocked the binding of SIRPα to CD47-expressing Raji-WT cells and Raji-GPC3^{H} cells to the same extent—GPC3/CD47 biAb exerts stronger blockade activity for Raji-GPC3^{H} cells than for unmodified Raji cells (FIG. 12D). Collectively, these results indicate that GPC3/CD47 biAb readily achieves preferential binding to, and effective SIRPα/CD47 blockade of, dual antigen-expressing tumor cells.

GPC3/CD47 biAb has a Superior Safety Profile and Extended Serum Half-Life in Human CD47/SIRPα Gene-Modified Mice Compared with Anti-CD47 mAb

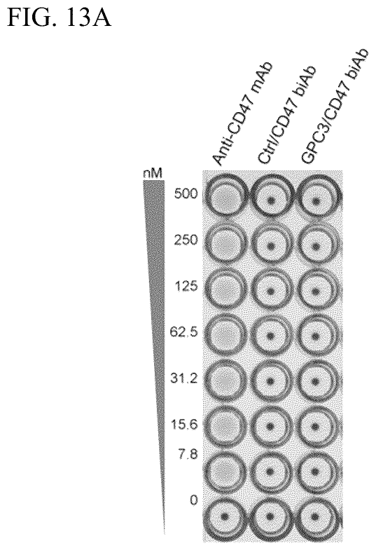

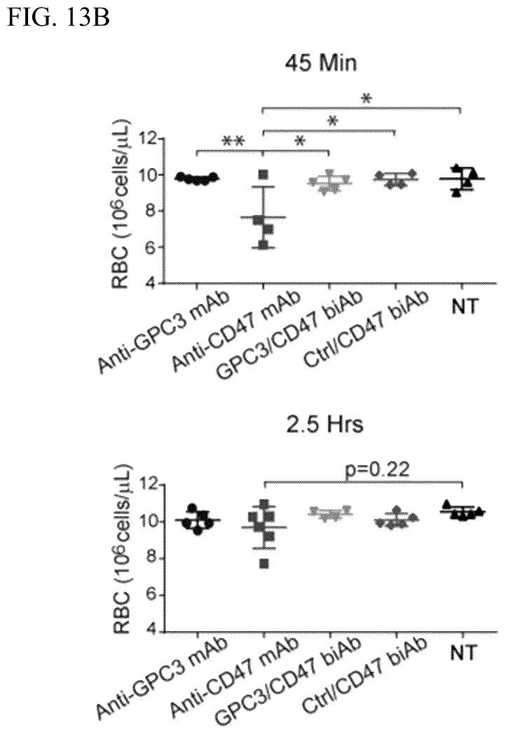

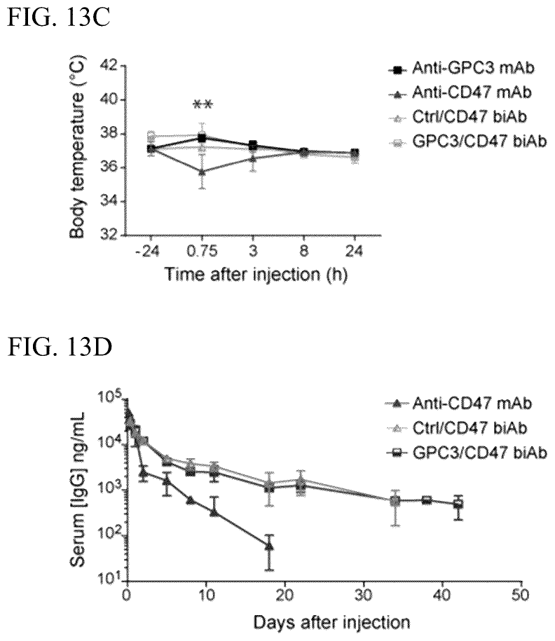

Severe side effects represent one of the major concerns for anti-CD47 antibody treatment. To evaluate the hematologic safety of our GPC3/CD47 biAb, we first performed in vitro agglutination assays with human red blood cells (RBCs). Anti-CD47 mAb caused hemagglutination as expected, whereas GPC3/CD47 biAb did not induce hemagglutination. In line with this, no hemagglutination occurred with Ctrl/CD47 biAb, indicating that binding to CD47 with only one antibody arm avoids induction of red blood cell agglutination (FIG. 13A).

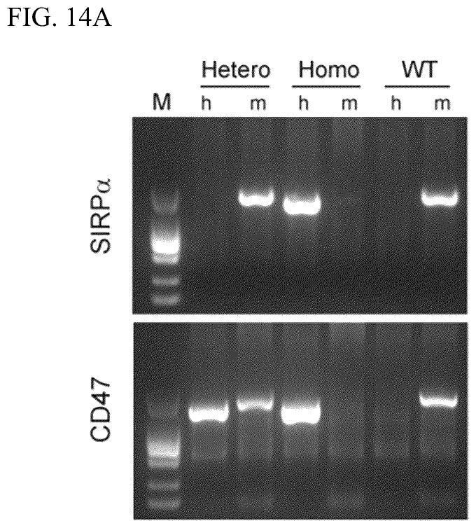

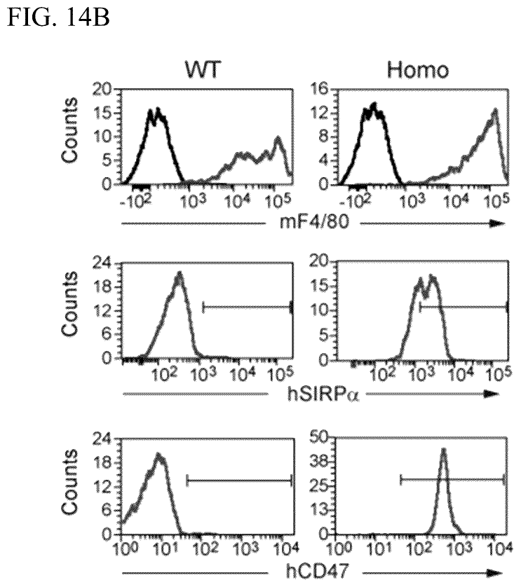

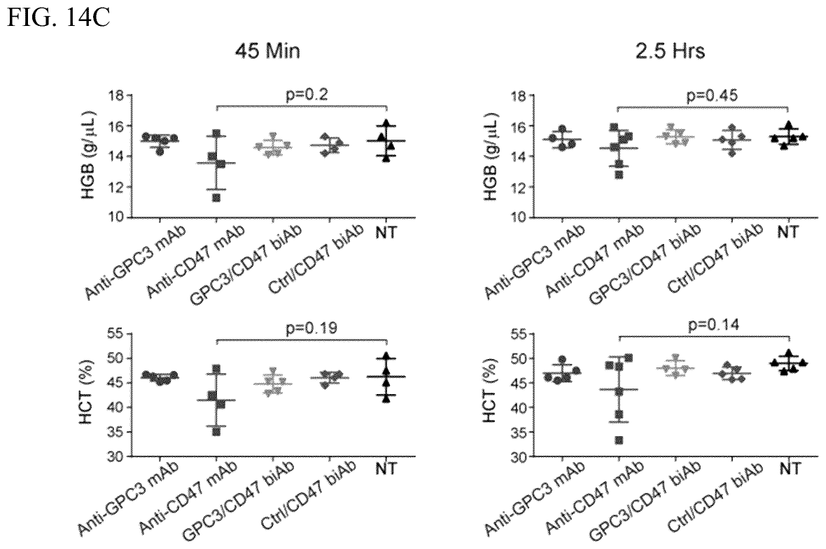

To assess the safety profile of GPC3/CD47 biAb in vivo, we generated human Cd47/Sirpα double gene-modified mice (hCD47/hSIRPα humanized mice), specifically the exons encoding the extracellular domains of murine Cd47 and Sirpα genes were replaced with human counterparts (FIG. 14A-B). No obvious signs of hematologic toxicity were observed for the biAbs, whereas anti-CD47 mAb induced acute red blood cell depletion within 1 hr in the humanized mice (FIG. 13B, FIG. 14C). Additionally, anti-CD47 mAb caused a significant drop in body temperature; no such drop occurred upon treatment with either the GPC3/CD47 or Ctrl/CD47 biAbs (FIG. 13C).

CD47-mediated rapid drug clearance is also a concern for clinical applications. We therefore performed a single-dose PK study with the aforementioned humanized mice. Similar to Ctrl/CD47 biAb, GPC3/CD47 biAb showed an extended serum half-life (t_{1/2}˜13.4 days) compared to the parental anti-CD47 mAb (t½˜2.4 days) (FIG. 13D). These extensions likely result from reduced antibody binding to normal somatic cells that express CD47 (antigen sink). Collectively, these results support that GPC3/CD47 biAb is apparently safer than anti-CD47 mAb and has a favorable PK profile in vivo.

GPC3/CD47 biAb Exerts Enhanced Fc-Mediated Functions and Selective Growth Suppression Against Dual Antigen-Expressing Tumors

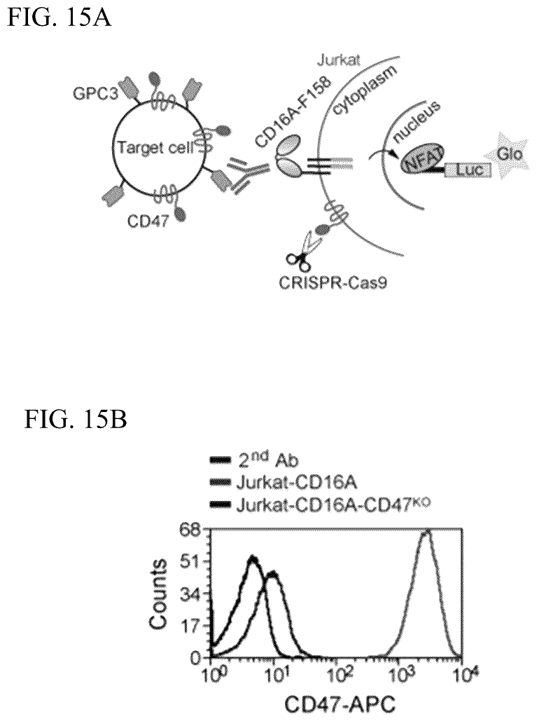

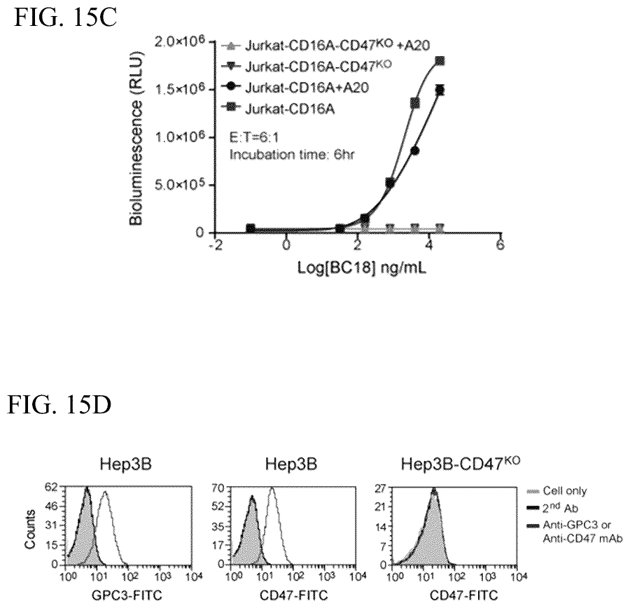

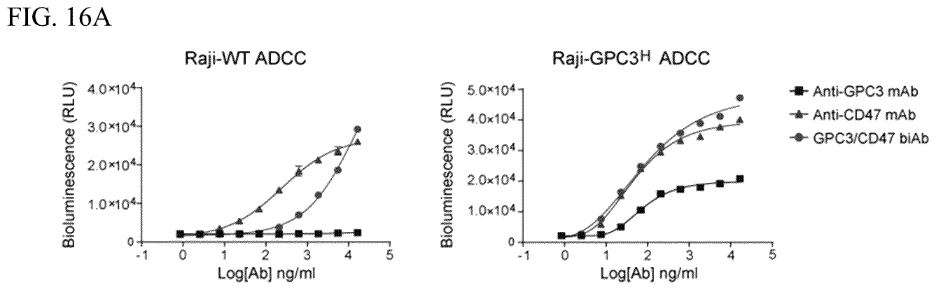

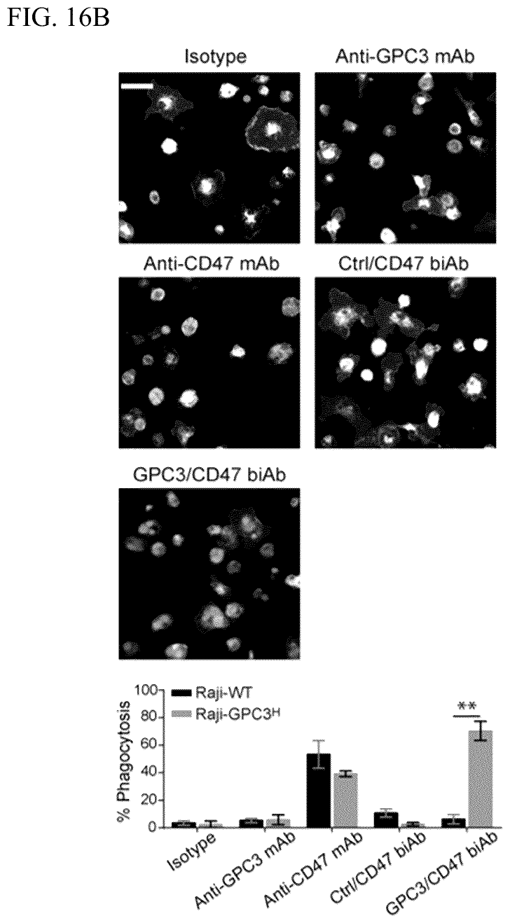

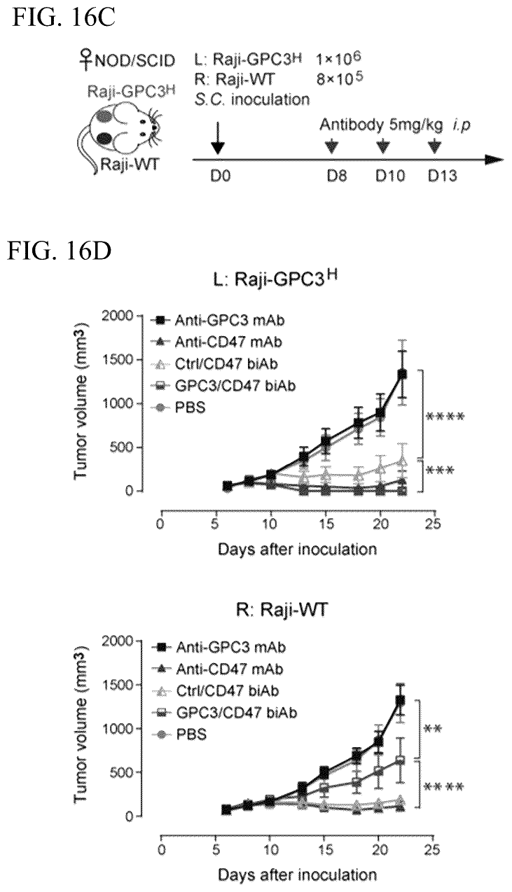

Antibody-dependent cellular cytotoxicity (ADCC) and antibody-dependent cellular phagocytosis (ADCP) mediated by the Fc domain are two functions elicited by antibodies during cancer therapy. In light of our finding that GPC3/CD47 biAb can preferentially bind to dual antigen-expressing tumor cells (FIG. 12C), we investigated GPC3/CD47 biAb's ability to induce ADCC and ADCP against Raji-GPC3^{H} cells. For the ADCC assays, we used a reporter system in which engineered Jurkat T lymphocyte cells were used as effector cells (FIG. 15A). We also knocked out CD47 expression to overcome a background toxicity issue caused by CD47 expression in Jurkat cells (Jurkat-CD16A-CD47KO) (FIG. 15B-C). Compared to anti-CD47 mAb, which induced equally strong ADCC against Raji-GPC3^{H} and Raji cells, GPC3/CD47 biAb induced stronger ADCC against Raji-GPC3H cells than Raji cells, doing so in a dose-dependent manner (FIG. 16A).

We then analyzed biAb-induced ADCP of Raji-GPC3^{H} cells upon co-incubation with macrophages in vitro. In this experiment, bone marrow derived macrophages (BMDMs) expressing human SIRPα from the humanized mice were used as effector cells, while a mixture of Raji-GPC3^{H} and Raji cells was used as target cells. Fluorescence microscopy and quantification of phagocytosis showed that GPC3/CD47 biAb induced preferential phagocytosis of Raji-GPC3^{H} cells (FIG. 16B). In contrast, the anti-CD47 mAb exhibited no selectivity: it induced phagocytosis of both types of cells.

To examine the antitumor efficacy of GPC3/CD47 biAb against dual antigen-expressing tumors during in vivo treatment, we used a bilateral mouse model wherein Raji or Raji-GPC3^{H} cells were respectively implanted in the right or left flank of NOD-SCID mice (FIG. 16C). Encouragingly, and in contrast to both anti-CD47 mAb and Ctrl/CD47 biAb—which each suppressed Raji-GPC3^{H} and Raji tumor progression at similar levels—GPC3/CD47 biAb's tumor suppressing effects were obviously more pronounced for Raji-GPC3H than for Raji cells (FIG. 16D). Anti-GPC3 mAb did not suppress Raji-GPC3H tumor progression, likely owing to its weak effector functions. Collectively, these in vitro and in vivo results show that GPC3/CD47 biAb exerts enhanced Fc-mediated functions and selective growth suppression against dual antigen-expressing tumors.

GPC3/CD47 biAb Outperforms Monotherapies and an Anti-CD47 and Anti-GPC3 mAbs Combination Therapy in a Xenograft HCC Model

To test the performance of GPC3/CD47 biAb on HCC progression in vivo, we used a GPC3 and CD47 double positive human HCC cell line (Hep3B) in a xenograft mouse model. After confirming that Hep3B cells express both GPC3 and CD47 at moderate levels (FIG. 15D), we established a Hep3B-Luc23 cell line stably expressing luciferase to enable measurement of tumor growth using in vivo bioluminescence imaging. NOD-SCID mice were subcutaneously injected with these Hep3B-Luc23 cells and were then randomized into four groups with similar mean tumor bioluminescence intensities prior to receiving antibody treatment twice each week for three weeks.

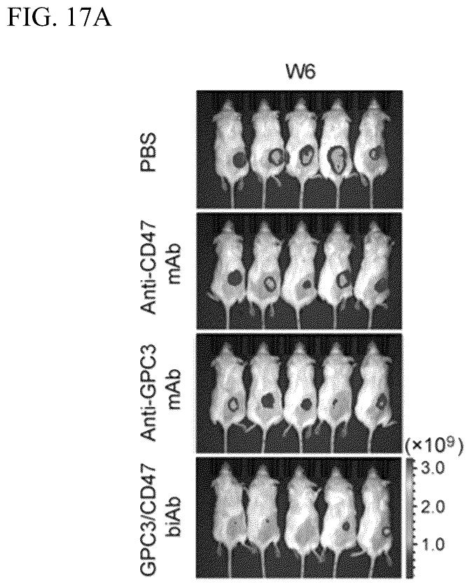

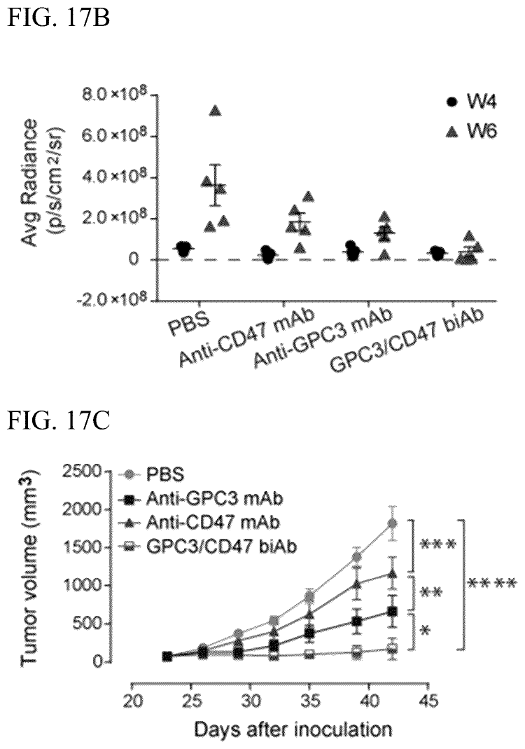

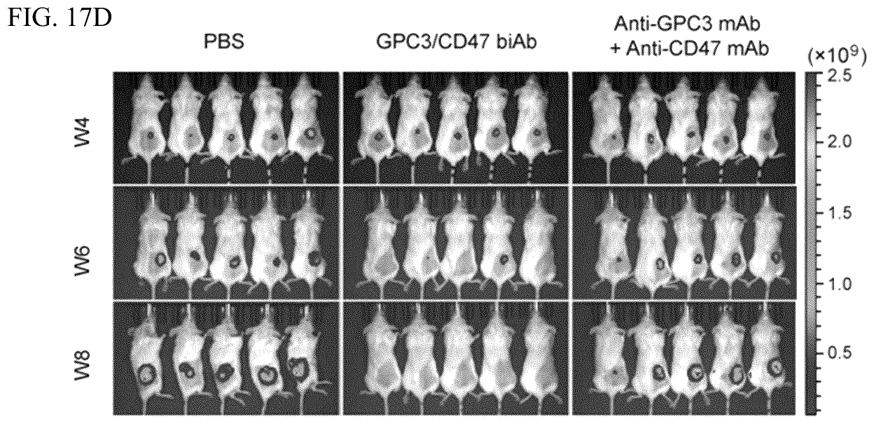

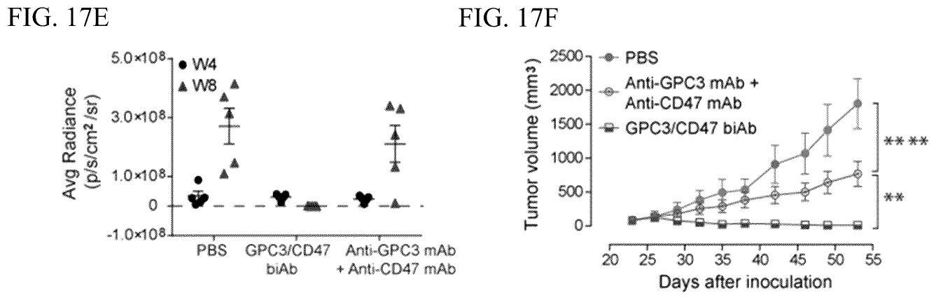

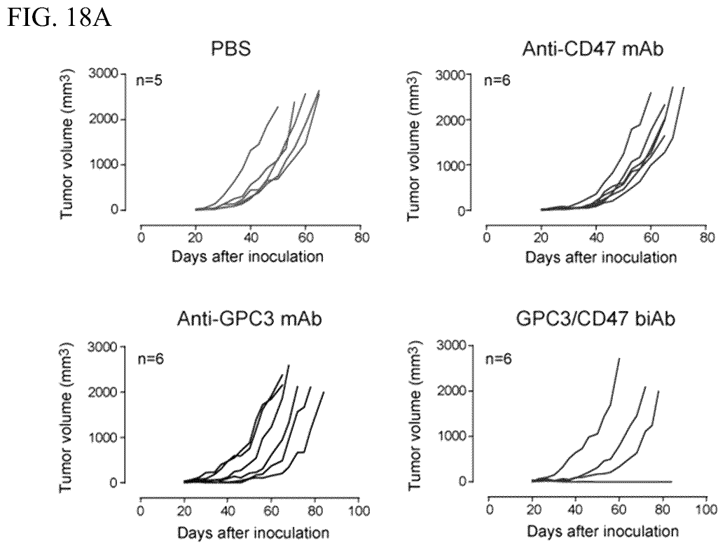

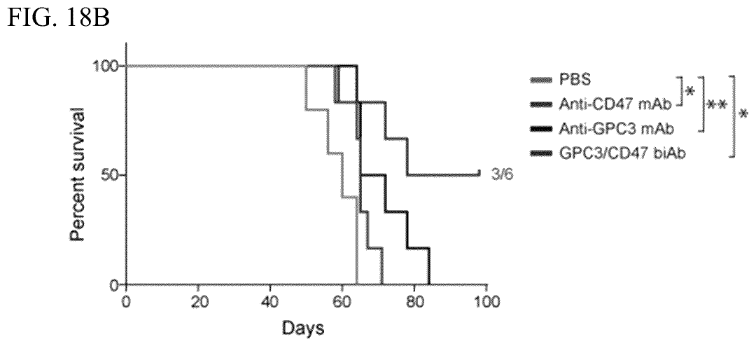

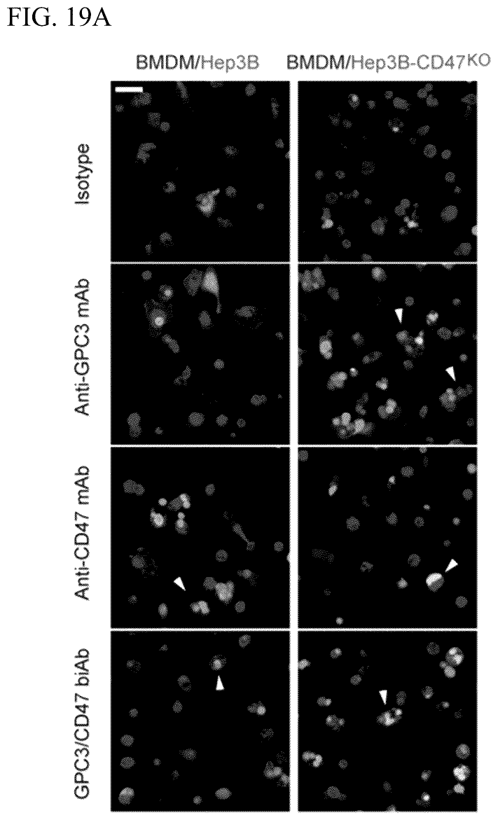

GPC3/CD47 biAb conferred significantly enhanced tumor suppression as compared to either anti-GPC3 mAb or anti-CD47 mAb, although it should be noted that each of these mAb monotherapies also inhibited Hep3B tumor growth. Bioluminescence imaging revealed complete eradication of the tumors in three of the five GPC3/CD47 biAb group mice; whereas no tumor eradication was detected for mice of the mAbs monotherapy groups (FIG. 17A-C). Using a similar dosing regimen, we also compared the effects of each antibody on the overall survival of tumor-bearing mice. Although both anti-CD47 mAb and anti-GPC3 mAb significantly increased the overall survival compared to PBS controls, all mice eventually died due to tumor progression. In contrast, GPC3/CD47 biAb elicited robust antitumor responses, resulting in complete response (CR) in 50% of mice (3 out of 6) for 2 months after the end of the treatment (FIG. 18A). GPC3/CD47 biAb treatment extended median survival to 88 days compared to a median survival of 65 days in the anti-CD47 mAb group and 68.5 days in the anti-GPC3 mAb group (FIG. 18B). We next compared the antitumor performance of GPC3/CD47 biAb against a combination therapy comprising anti-GPC3 mAb and anti-CD47 mAb. Monitoring of tumor growth over time by measuring tumor volumes and bioluminescence intensities showed that GPC3/CD47 biAb conferred superior antitumor effects compared to the combination therapy (FIG. 17D-F). Taken together, these results provide a powerful demonstration of the antitumor activity of GPC3/CD47 biAb against GPC3 and CD47 double positive HCC tumors.

Macrophages and Neutrophils are Involved in GPC3/CD47 biAb's Fc-Dependent Antitumor Activities In Vivo

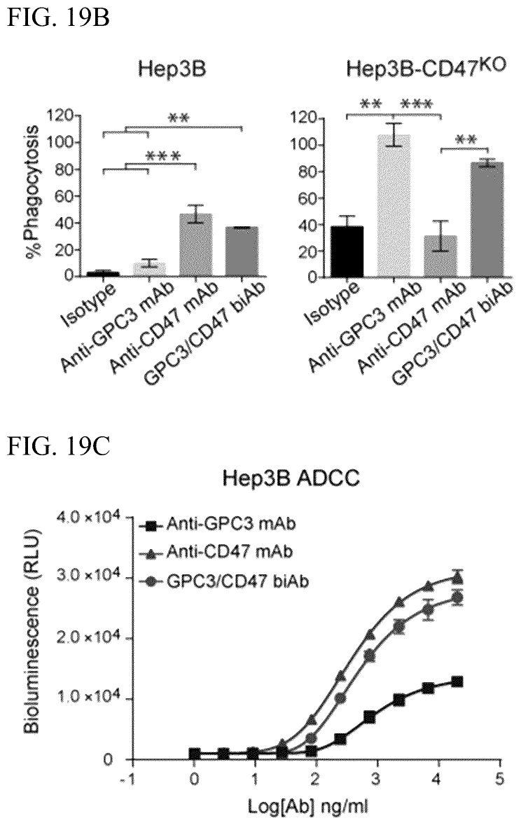

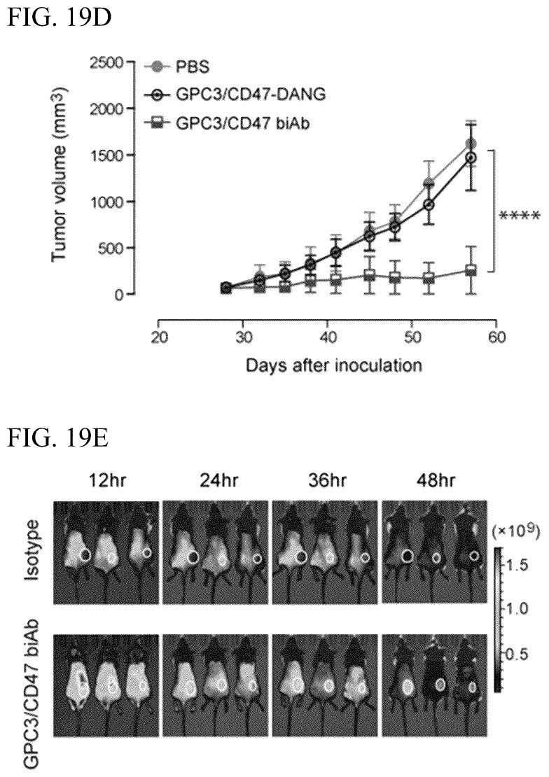

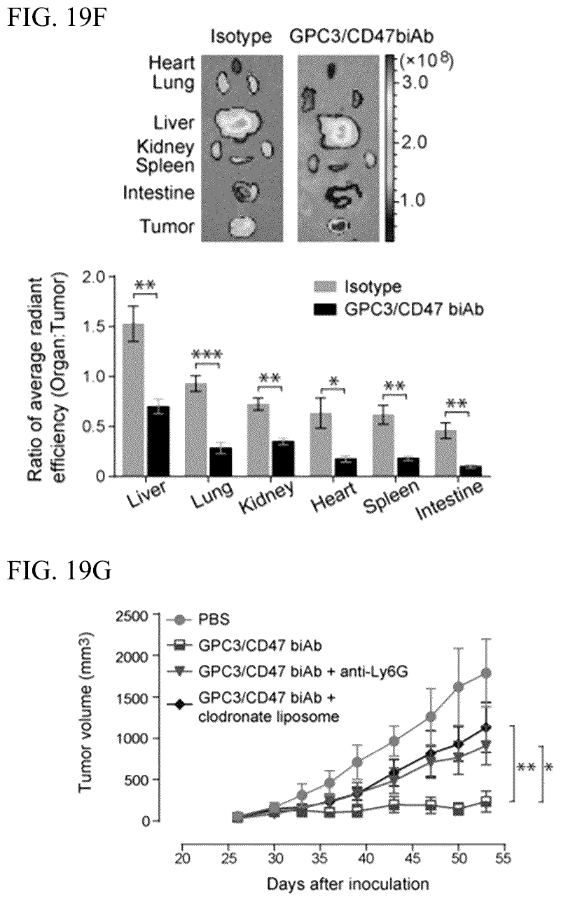

To investigate GPC3/CD47 biAb's mechanism of action for treatment of HCC, we first examined Fc-mediated effector functions against Hep3B cells. In vitro ADCP experiments showed that GPC3/CD47 biAb induced significant phagocytosis of Hep3B cells. We also found that anti-GPC3 mAb did not induce phagocytosis of Hep3B cells, whereas genetic knockout of CD47 from Hep3B cells (Hep3B-CD47^{KO}) restored the phagocytic activity of this anti-GPC3 mAb (FIG. 19A-B), a finding that confirms previous reports about CD47 expression on tumor cells conferring resistance to antibody-induced phagocytosis. In vitro ADCC assays, GPC3/CD47 biAb induced dose-dependent cytotoxic effects against Hep3B cells at a level comparable to or higher than the anti-CD47 or anti-GPC3 mAbs (FIG. 19C).

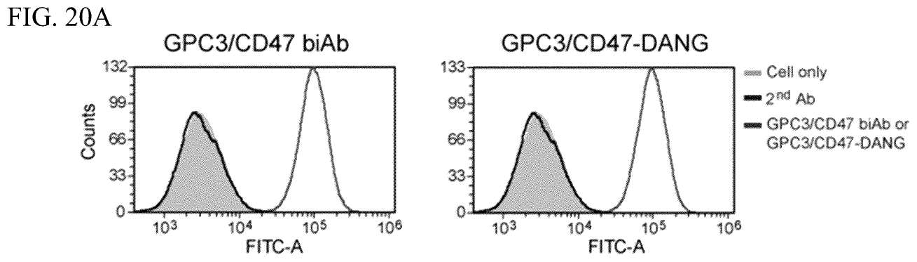

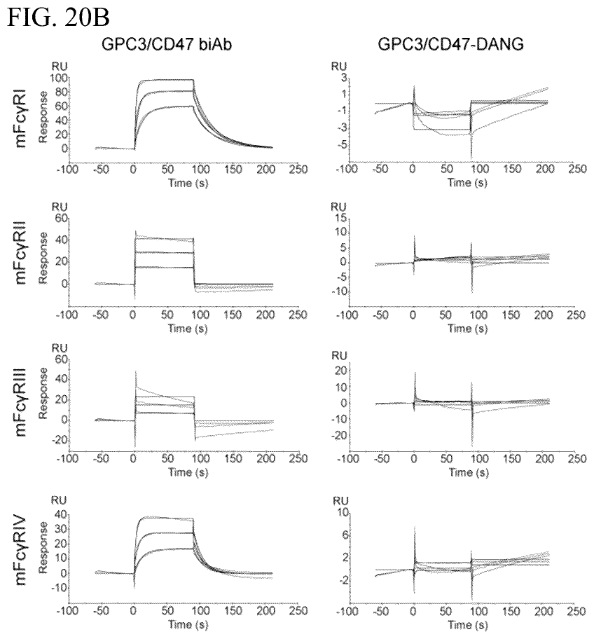

To further assess Fc-mediated effector functions in vivo, we generated a GPC3/CD47 biAb variant (GPC3/CD47-DANG) bearing two mutations (D265A and N297G) in its Fc region that are known to abolish the binding of Fc regions with all classes of FcγRs. Prior to conducting mouse studies, we successfully confirmed that GPC3/CD47-DANG exhibited no binding activity for mFcγRs yet retained its capacity to bind to Hep3B cells, doing so with similar affinity as GPC3/CD47 biAb (FIG. 20A-B). In experiments using the aforementioned Hep3B-luc xenograft model, we found that GPC3/CD47-DANG conferred no antitumor effects (FIG. 19D), indicating that Fc-mediated effector functions are required for biAb's antitumor activity. Using whole-body near-infrared fluorescence (NIRF) imaging, we also assessed the distribution of GPC3/CD47 biAb in tumor-bearing mice. GPC3/CD47 biAb exhibited increased tumor accumulation over time as compared to the isotype control antibody (FIG. 19E). To measure the accumulation of each antibody in different organs, the fluorescence signals from freshly dissected tumors and organs were quantified. In contrast to the apparently nonspecific distribution of the isotype control antibody, the significantly decreased organ to tumor fluorescence ratios in GPC3/CD47 biAb treated mice supported its specific accumulation at the tumor site (FIG. 19F).

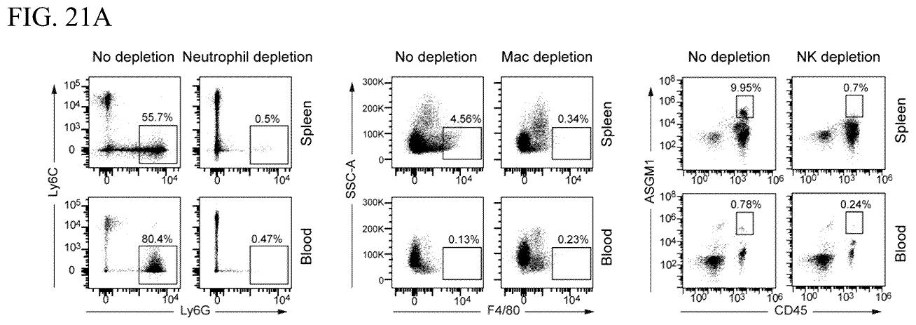

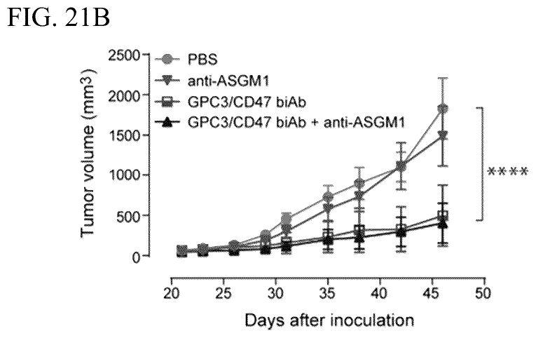

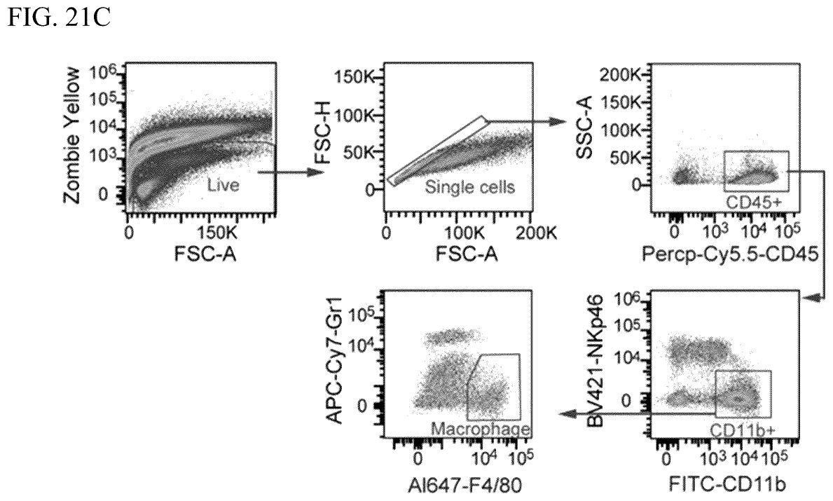

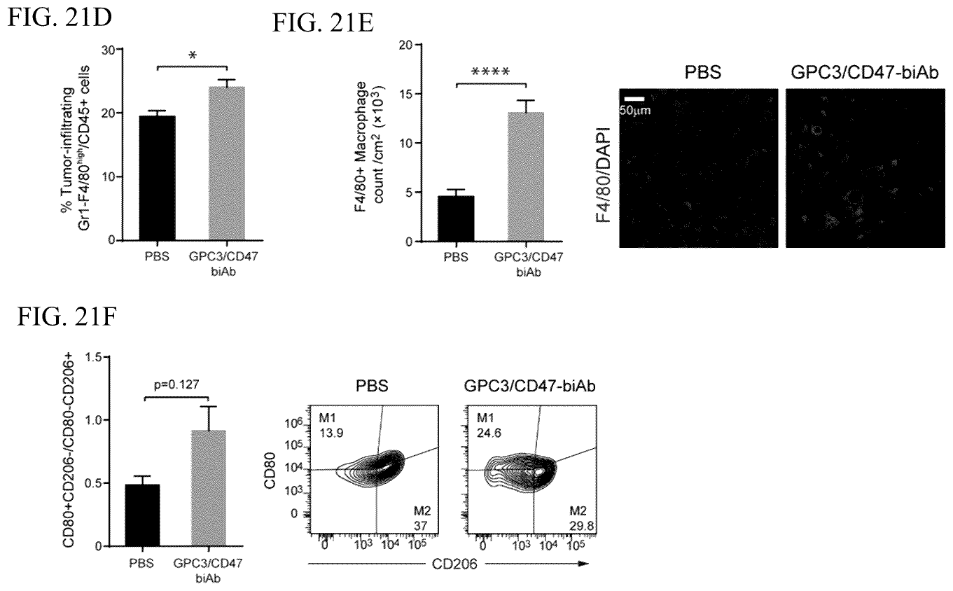

We next sought to identify the immune cell types that contribute to the antitumor effects of GPC3/CD47 biAb. Given the immunocompromising immune background of NOD-SCID mice, we used an immune cell depletion method to assess the contribution of the model's three major innate immune cell subsets: macrophages, neutrophils, and NK cells. The depletion efficiencies were validated to be over 90% for all three immune cell subsets (FIG. 21A). Depletion of either macrophages or neutrophils from mice led to significant reductions in GPC3/CD47 biAb's antitumor effects, whereas depletion of NK cells did not cause any effect (FIG. 19G, FIG. 20B). This result supports that both macrophages and neutrophils functionally contribute to biAb's antitumor effects.

In light of previous studies showing that GPC3 overexpression in HCC may recruit M2-polarized immunosuppressive macrophages—an outcome thought to limit the treatment efficacy of antibodies—we further analyzed the infiltrated macrophages after GPC3/CD47 biAb treatment. Compared to the vehicle control, FACS analysis revealed that the frequency of infiltrated macrophages was significantly elevated in mice treated with GPC3/CD47 biAb (FIG. 21C-D). This result was further supported by results from an immunofluorescence microscopy analysis (FIG. 21E). Additionally, GPC3/CD47 biAb treatment also appeared to cause an increased M1-like/M2-like ratio, a finding suggesting that GPC3/CD47 biAb can somehow bias intratumoral macrophages towards a pro-inflammatory status (FIG. 21F). Viewed together, these results from NOD-SCID xenograft tumor models establish that GPC3/CD47 biAb's antitumor activity requires both its Fc-mediated effector functions and the involvement of macrophages and neutrophils.

FIG. 21:(A) In vivo depletion of macrophages, neutrophils, and NK cells. NOD-SCID mice were i.p. injected with 200 μL clodronate liposome (FormuMax) three days prior to initiation of GPC3/CD47 biAb treatment, followed by additional injection (100 μL) once a week until the end of the study. Neutrophils were depleted using 200 μg anti-Ly6G antibody (1A8 clone, BioXCell) every five days. NK cells were depleted using 50 μL anti-Asialo-GM1 polyclonal antibodies (Poly21460, BioLegend) once a week. The depletion efficacy in spleen and peripheral blood was confirmed by flow cytometry. (B) In vivo tumor growth after GPC3/CD47 biAb treatment with or without the depletion of NK cells (anti-ASGM1). Tumor volumes were calculated based on caliper measurements and are shown as the mean±SEM (****p<0.0001; 2-way ANOVA followed by Tukey's multiple comparisons test). (C) Flow cytometry gating strategy to identify tumor infiltrating macrophages from CD45⁺ immune cells; the CD45⁺ cells were initially gated after the exclusion of dead cells and doublets. Intratumoral macrophages were defined as CD11b⁺Gr-1⁻F4/80^{high}. (D) Percentage of tumor infiltrating macrophages among CD45⁺ immune cells by FACS. (E) IHC analysis of tumor infiltrating F4/80+ cell counts (left) and representative F4/80 staining tumor images with or without GPC3/CD47 biAb treatment (right, magnification ×40). (F) Bar chart depicting the M1-like/M2-like macrophage ratio (left) and representative FACS plots showing M1-like and M2-like subsets based on CD80 and CD206 staining (right).

### DISCUSSION

Despite numerous advances in cancer therapy in recent years, there is still a large proportion of patients with advanced HCC who do not respond to first-line treatment or to immune checkpoint inhibitors. Therefore, development of a treatment strategy that exploits distinct mechanisms would likely greatly benefit HCC patients. Bispecific antibodies represent an emerging class of antibody therapies which have been explored for treating a variety of tumor types. In the present study, we generated a bispecific antibody that binds to GPC3 and CD47 to specifically target HCC tumor cells, and we assessed the pharmacokinetics and potential adverse effects of human-CD47-targeting bispecific antibodies in hCD47/hSIRPα double humanized mice. GPC3/CD47 biAb has an attractive half-life and displayed no hematological toxicity. We also exploited a bilateral mouse model to empirically establish the preferential activity of GPC3/CD47 biAb for tumor cells over normal cells. Collectively, the safety profile, long half-life, and preferential antitumor activity of GPC3/CD47 biAb highlight its apparent advantages compared to combinational therapies that employ an anti-CD47 monoclonal antibody.

Recall that our in vitro ADCP experiments showed how anti-GPC3 mAb exerts significant phagocytosis of Hep3B after CD47 knockout. This result corroborates previous studies reporting that CD47 expression on tumor cells can hamper the pro-phagocytic potential of tumor-antigen-targeted therapeutic antibodies. Therefore, our work illustrates how the exploitation of CD47 blockade can add an additional pro-phagocytic antitumor response for the treatment of CD47 positive HCC. Tumor associated macrophages (TAMs) display a continuum of different polarization states between antitumorigenic M1 and protumorigenic M2 phenotypes. Previous studies showed that elevated M2 macrophage levels in tumors are associated with poor prognosis for HCC. In addition to activating macrophage phagocytosis, GPC3/CD47 biAb also improved the ratio of M1/M2 macrophages. It is plausible that this circumstance may be additionally favorable for HCC patients.

In contrast to the well-studied antitumor activities of macrophages, there is relatively limited evidence that neutrophils mediate antitumor effects. Although neutrophils—which express Fc receptors—represent the most abundant population of circulating cytotoxic effector cells in human, their exact mechanism(s) for killing antibody-opsonized cancer cells remain elusive. Intriguingly, our results support that neutrophils are required for the antitumor effects of GPC3/CD47 biAb in NOD-SCID mice. Similar to our findings, previous studies have shown that selective depletion of neutrophils significantly reduces the protective activity of an anti-CD52 mAb and an anti-CD20 mAb. Blockade of the CD47-SIRPα interaction has been shown to augment antibody-mediated tumor cell killing by neutrophils. Interestingly, there are also reports of neutrophils involvement in HCC progression. Thus, it will be interesting to examine whether and how neutrophils contribute to GPC3/CD47 biAb's antitumor effects in HCC patients.

When considering the antitumor activity of GPC3/CD47 biAb in HCC patients, hepatic NK cells cannot be ignored. These cells, which account for up to 50% of total hepatic lymphocytes, are involved in the inhibition of viral infection as well as liver tumorigenesis. It is notable that many studies have observed impaired cytotoxicity and decreased infiltration of NK cells as HCC progresses. This reduction in NK cells in tumors would apparently limit the antitumor efficacy of ADCC-mediating antibodies. Previous studies have demonstrated the antitumor efficacy of ADCC-mediating therapeutic antibodies is affected by polymorphisms in FcγRIIIa (158V/F) expressed on NK cells. Moreover, it is notable that more than 85% of the human population are known to carry a low-affinity FcγRIIIa-158F allotype. The failure of a phase II clinical trial of anti-GPC3 mAb (GC33) in patients with advanced HCC can perhaps be attributed to sub-optimal ADCC activity; and selecting patients with high-affinity FcγRIIIa allotypes or high expression levels of GPC3 and/or FcγRIIIa may promote a high ADCC effect, which could ultimately improve outcomes. Taking this into consideration, and recalling GPC3/CD47 biAb's greatly enhanced ADCC activity as compared to the parental anti-GPC3 mAb against Hep3B cells (which express moderate levels of GPC3), we anticipate that an ability of GPC3/CD47 biAb to elicit enhanced ADCC of NK cells may promote its overall efficacy for treating a broader range of HCC patients (i.e., patients having heterogeneous GPC3 expression profiles). Although the NK-dysfunctional NOD-SCID mouse model of our study precluded the unambiguous confirmation of NK cells' involvement in GPC3/CD47 biAb's antitumor effects, close monitoring of NK cell activity in future preclinical and clinical trials of this antibody (including combination therapies) will very likely deepen our understanding of how ADCC activity can promote NK-mediated tumor killing.

It should be emphasized that other host immune cells such as T cells also contribute significantly in antitumor protection. Several studies have demonstrated therapeutic efficacy of GPC3-targeting CAR T cells and T cell-engaging bispecific antibodies in HCC xenograft models, but these presently meet numerous challenges in clinical practice, especially for solid tumors. It is noteworthy that CD47-blocking antibodies can harness antigen-presenting cells (macrophages or dendritic cells) and promote tumor antigen cross-presentation to T cells, which leads to durable adaptive antitumor responses. Thus, we assume that our GPC3/CD47 biAb may also exploit T cell antitumor activity, and its HCC therapeutic efficacy could potentially be further maximized when deployed as a combination agent alongside other T cell stimulating immune checkpoint inhibitors or chemotherapeutics. Future studies administering GPC3/CD47 biAb together with T cell immune checkpoint inhibitors such as anti-PD-1 or anti-CTLA-4 mAbs in immune-competent models are warranted to address their respective contributions.

It is interesting to note that, in addition to exhibiting much stronger antitumor activity than monoclonal antibodies alone, treatment with GPC3/CD47 biAb conferred superior antitumor benefit over a combination therapy comprising two antibodies that separately recognize the GPC3 and CD47 targets. This observation supports that the binding of two antigens using a bispecific antibody may offer improved clinical value through some form of synergism. In sum, we here developed a bispecific antibody and demonstrated its safe and efficient antitumor activity for HCC. Ultimately, as we learn more about the specific mechanisms of GPC3/CD47 biAb in treating HCC, we may uncover more general trends that can support the development of bispecific antibodies to boost innate immune responses for innovative therapeutic strategies to further improve cancer treatment outcomes.

1. An isolated anti-CD47 antibody or fragment thereof having the ability of binding CD47 and competing with the binding of SIRPa to CD47, comprising:
a heavy chain variable region comprising heavy chain complementarity determining regions HCDR1, HCDR2, and HCDR3; and
a light chain variable region comprising light chain complementarity determining regions LCDR1, LCDR2, and LCDR3, wherein:
each of HCDR1, HCDR2, and HCDR3 is selected from the group consisting of: (1) HCDR1 having the amino acid sequence of SEQ ID NO: 1, HCDR2 having the amino acid sequence of SEQ ID NO: 2, HCDR3 having the amino acid sequence of SEQ ID NO: 3; (2) HCDR1 having the amino acid sequence of SEQ ID NO: 4, HCDR2 having the amino acid sequence of SEQ ID NO: 5, HCDR3 having the amino acid sequence of SEQ ID NO: 6; (3) HCDR1 having the amino acid sequence of SEQ ID NO: 7, HCDR2 having the amino acid sequence of SEQ ID NO: 8, HCDR3 having the amino acid sequence of SEQ ID NO: 9; (4) HCDR1 having the amino acid sequence of SEQ ID NO: 10, HCDR2 having the amino acid sequence of SEQ ID NO: 11, HCDR3 having the amino acid sequence of SEQ ID NO: 12; (5) HCDR1 having the amino acid sequence of SEQ ID NO: 13, HCDR2 having the amino acid sequence of SEQ ID NO: 14, HCDR3 having the amino acid sequence of SEQ ID NO: 15; and (6) HCDR1, HCDR2, HCDR3 as shown in (1)-(5), but at least one of which includes one, two, three, four or five amino acids addition, deletion, conservative amino acid substitution or the combinations thereof; and
each of LCDR1, LCDR2, and LCDR3 is selected from the group consisting of:
(1) LCDR1 having the amino acid sequence of SEQ ID NO: 16, LCDR2 having the amino acid sequence of SEQ ID NO: 17, LCDR3 having the amino acid sequence of SEQ ID NO: 18; (2) LCDR1 having the amino acid sequence of SEQ ID NO: 19, LCDR2 having the amino acid sequence of SEQ ID NO: 20, LCDR3 having the amino acid sequence of SEQ ID NO: 21; (3) LCDR1 having the amino acid sequence of SEQ ID NO: 22, LCDR2 having the amino acid sequence of SEQ ID NO: 23, LCDR3 having the amino acid sequence of SEQ ID NO: 24; (4) LCDR1 having the amino acid sequence of SEQ ID NO: 25, LCDR2 having the amino acid sequence of SEQ ID NO: 26, LCDR3 having the amino acid sequence of SEQ ID NO: 27; (5) LCDR1 having the amino acid sequence of SEQ ID NO: 28, LCDR2 having the amino acid sequence of SEQ ID NO: 29, LCDR3 having the amino acid sequence of SEQ ID NO: 30; and (6) LCDR1, LCDR2, LCDR3 as shown in (1)-(5), but at least one of which includes one, two, three, four or five amino acids addition, deletion, conservative amino acid substitution or the combinations thereof.

2. The isolated anti-CD47 antibody or fragment thereof of claim 1, which is a human anti-CD47 antibody or fragment thereof.

3. (canceled)

4. The isolated anti-CD47 antibody or fragment thereof of claim 1, wherein:
each of HCDR1, HCDR2, HCDR3, LCDR1, LCDR2, and LCDR3 is selected from the group consisting of: (1) HCDR1 having the amino acid sequence of SEQ ID NO: 1, HCDR2 having the amino acid sequence of SEQ ID NO: 2, HCDR3 having the amino acid sequence of SEQ ID NO: 3, LCDR1 having the amino acid sequence of SEQ ID NO: 16, LCDR2 having the amino acid sequence of SEQ ID NO: 17, LCDR3 having the amino acid sequence of SEQ ID NO: 18; (2) HCDR1 having the amino acid sequence of SEQ ID NO: 4, HCDR2 having the amino acid sequence of SEQ ID NO: 5, HCDR3 having the amino acid sequence of SEQ ID NO: 6, LCDR1 having the amino acid sequence of SEQ ID NO: 19, LCDR2 having the amino acid sequence of SEQ ID NO: 20, LCDR3 having the amino acid sequence of SEQ ID NO: 21; (3) HCDR1 having the amino acid sequence of SEQ ID NO: 7, HCDR2 having the amino acid sequence of SEQ ID NO: 8, HCDR3 having the amino acid sequence of SEQ ID NO: 9, LCDR1 having the amino acid sequence of SEQ ID NO: 22, LCDR2 having the amino acid sequence of SEQ ID NO: 23, LCDR3 having the amino acid sequence of SEQ ID NO: 24; (4) HCDR1 having the amino acid sequence of SEQ ID NO: 10, HCDR2 having the amino acid sequence of SEQ ID NO: 11, HCDR3 having the amino acid sequence of SEQ ID NO: 12, LCDR1 having the amino acid sequence of SEQ ID NO: 25, LCDR2 having the amino acid sequence of SEQ ID NO: 26, LCDR3 having the amino acid sequence of SEQ ID NO: 27; (5) HCDR1 having the amino acid sequence of SEQ ID NO: 13, HCDR2 having the amino acid sequence of SEQ ID NO: 14, HCDR3 having the amino acid sequence of SEQ ID NO: 15, LCDR1 having the amino acid sequence of SEQ ID NO: 28, LCDR2 having the amino acid sequence of SEQ ID NO: 29, LCDR3 having the amino acid sequence of SEQ ID NO: 30; and (6) HCDR1, HCDR2, HCDR3, LCDR1, LCDR2, LCDR3 as shown in (1)-(5), but at least one of which includes one, two, three, four or five amino acids addition, deletion, conservative amino acid substitution or the combinations thereof.

5. The isolated anti-CD47 antibody or fragment thereof of claim 1, wherein the heavy chain variable region has the amino acid sequence selected from the group consisting of: the amino acid sequences shown in SEQ ID NOs: 37-41, and an amino acid sequence having at least 95% sequence identity to any one of the amino acid sequences shown in SEQ ID NOs: 37-41, and retaining the activity of epitope-binding, and
wherein the light chain variable region has the amino acid sequence selected from the group consisting of: the amino acid sequences shown in SEQ ID NOs: 42-46, and an amino acid sequence having at least 95% sequence identity to any one of the amino acid sequences shown in SEQ ID NOs: 42-46, and retaining the activity of epitope-binding.

6. The isolated anti-CD47 antibody or fragment thereof of claim 1, wherein the heavy chain variable region and the light chain variable region have the amino acid sequences selected from the group consisting of:
(1) the amino acid sequence shown in SEQ ID NO: 37, and the amino acid sequence shown in SEQ ID NO: 42; (2) the amino acid sequence shown in SEQ ID NO: 38, and the amino acid sequence shown in SEQ ID NO: 43; (3) the amino acid sequence shown in SEQ ID NO: 39, and the amino acid sequence shown in SEQ ID NO: 44; (4) the amino acid sequence shown in SEQ ID NO: 40, and the amino acid sequence shown in SEQ ID NO: 45; (5) the amino acid sequence shown in SEQ ID NO: 41, and the amino acid sequence shown in SEQ ID NO: 46; and (6) two amino acid sequences having at least 95% sequence identity to any one of (1)-(5) respectively, and retaining the activity of epitope-binding.

7. A composition comprising the isolated anti-CD47 antibody or fragment thereof of claim 1 and a pharmaceutical acceptable carrier.

8. A method for treating a disorder in which CD47 is overexpressed or upregulated in a subject, comprising:
using an isolated anti-CD47 antibody or fragment thereof having the ability of binding CD47 and competing with the binding of SIRPa to CD47.

9. The method of claim 8, wherein the disorder is a cancer selected from the group consisting of: solid tumor cancers of the lung, prostate, breast, bladder, colon, ovarian, pancreas, kidney, liver, glioblastoma, medulloblastoma, leiomyosarcoma, head and neck squamous cell carcinomas, melanomas, liquid cancers, hematological cancers, leukemias, lymphomas, brain cancers, an infection, a chronic infection, an immunological disease, an immunological disorder, an inflammatory disease, multiple sclerosis, and arthritis.

10. (canceled)

11. A bispecific antibody, comprising:
a first antigen binding moiety that binds to human GPC3 (hGPC3); and
a second antigen binding moiety that binds to human CD47 (hCD47).

12. The bispecific antibody of claim 11, wherein the first antigen binding moiety that binds to human GPC3 comprises:
(a) a VH domain comprising (i) HCDR1 having the amino acid sequence of SEQ ID NO: 31, (ii) HCDR2 having the amino acid sequence of SEQ ID NO: 32, and (iii) HCDR3 having the amino acid sequence of SEQ ID NO: 33; and
(b) a VL domain comprising (i) LCDR1 having the amino acid sequence of SEQ ID NO: 34, (ii) LCDR2 having the amino acid sequence of SEQ ID NO: 35, and (iii) LCDR3 having the amino acid sequence of SEQ ID NO: 36.

13. The bispecific antibody of claim 11, wherein the first antigen binding moiety that binds to human GPC3 comprises:
(a) a VH domain comprising the amino acid sequence of SEQ ID NO: 47; and
(b) a VL domain comprising the amino acid sequence of SEQ ID NO: 48.

14. The bispecific antibody of claim 11, wherein the second antigen binding moiety that binds to human CD47 comprises:
(a) a VH domain comprising (i) HCDR1 having the amino acid sequence of SEQ ID NO: 1, (ii) HCDR2 having the amino acid sequence of SEQ ID NO: 2, and (iii) HCDR3 having the amino acid sequence of SEQ ID NO: 3; and
(b) a VL domain comprising (i) LCDR1 having the amino acid sequence of SEQ ID NO: 16, (ii) LCDR2 having the amino acid sequence of SEQ ID NO: 17, and (iii) LCDR3 having the amino acid sequence of SEQ ID NO: 18.

15. The bispecific antibody of claim 11, wherein the first antigen binding moiety that binds to human CD47 comprises:
(a) a VH domain comprising the amino acid sequence of SEQ ID NO: 37; and
(b) a VL domain comprising the amino acid sequence of SEQ ID NO: 42.

16. The bispecific antibody of claim 11, wherein the bispecific antibody further comprises a Fc region having a Hinge portion, a CH3 portion and a CH2 portion.

17. (canceled)

18. The bispecific antibody of claim 16, wherein the Fc region further comprises domains that promote heterodimerization, wherein the Fc region includes a knob domain and a hole domain that allow for heterodimerization of the two heavy chains, wherein the knob domain and the hole domain are positioned in CH3 portions respectively.

19. The bispecific antibody of claim 18, wherein the knob domain has the knob mutations T366W and S354C, and the hole domain has the hole mutations Y407V, L368A, T366S and Y349C.

20. The bispecific antibody of claim 11, which has a CH1 portion and a CL portion, wherein the CH1 portion and the CL portion are replaced by each other.

21. The bispecific antibody of claim 11, which comprises a knob chain (HC1 chain) and a hole chain (HC2 chain), wherein the knob chain comprises the first antigen binding moiety, CH1 portion, hinge portion, CH2 portion and CH3 region, and the hole chain comprises the second antigen binding moiety, CL portion, Hinge portion, CH2 portion and CH3 portion, wherein the bispecific antibody further comprises a LC1 portion comprising VL and CL, and a LC2 portion comprising VL and CH1.

22. The bispecific antibody of claim 11, which comprises a HC1 portion having the amino acid sequence of SEQ ID NO: 49, a LC1 portion having the amino acid sequence of SEQ ID NO: 50, HC2 having the amino acid sequence of SEQ ID NO: 51, and a LC2 portion having the amino acid sequence of SEQ ID NO: 52, wherein HC1 and HC2 are interlinked by three disulfide bonds and in a knob-into-hole way, and LC1 and LC2 are attached to HC1 and HC2, respectively, by a disulfide bond.

23. A method for treating hepatocellular carcinoma (HCC) comprising the use of the bispecific antibody of claim 11.


SEQUENCE LISTING (52 sequences)

SEQ ID NO: 1 - protein, 5 aa, Artificial Sequence
  The sequence is synthesized.
YHYWS

SEQ ID NO: 2 - protein, 16 aa, Artificial Sequence
  The sequence is synthesized.
YIYYSGSTTYNPSLKS

SEQ ID NO: 3 - protein, 7 aa, Artificial Sequence
  The sequence is synthesized.
QRGAYDY

SEQ ID NO: 4 - protein, 5 aa, Artificial Sequence
  The sequence is synthesized.
YHYWS

SEQ ID NO: 5 - protein, 16 aa, Artificial Sequence
  The sequence is synthesized.
YIYYSGSTTYNPSLKS

SEQ ID NO: 6 - protein, 7 aa, Artificial Sequence
  The sequence is synthesized.
QRGAYDY

SEQ ID NO: 7 - protein, 5 aa, Artificial Sequence
  The sequence is synthesized.
YHYWS

SEQ ID NO: 8 - protein, 16 aa, Artificial Sequence
  The sequence is synthesized.
YIYYSGSTTYNPSLKS

SEQ ID NO: 9 - protein, 7 aa, Artificial Sequence
  The sequence is synthesized.
QRGAYDY

SEQ ID NO: 10 - protein, 5 aa, Artificial Sequence
  The sequence is synthesized.
YHYWS

SEQ ID NO: 11 - protein, 16 aa, Artificial Sequence
  The sequence is synthesized.
YIYYSGSTTYNPSLKS

SEQ ID NO: 12 - protein, 7 aa, Artificial Sequence
  The sequence is synthesized.
QRGAYDY

SEQ ID NO: 13 - protein, 5 aa, Artificial Sequence
  The sequence is synthesized.
YHYWS

SEQ ID NO: 14 - protein, 16 aa, Artificial Sequence
  The sequence is synthesized.
YIYYSGSTTYNPSLKS

SEQ ID NO: 15 - protein, 7 aa, Artificial Sequence
  The sequence is synthesized.
QRGAYDY

SEQ ID NO: 16 - protein, 11 aa, Artificial Sequence
  The sequence is synthesized.
RASQDIGSRLV

SEQ ID NO: 17 - protein, 7 aa, Artificial Sequence
  The sequence is synthesized.
AASSLQS

SEQ ID NO: 18 - protein, 9 aa, Artificial Sequence
  The sequence is synthesized.
QQSYSTPYS

SEQ ID NO: 19 - protein, 11 aa, Artificial Sequence
  The sequence is synthesized.
RASQDIGSRLV

SEQ ID NO: 20 - protein, 6 aa, Artificial Sequence
  The sequence is synthesized.
AASSLQ

SEQ ID NO: 21 - protein, 9 aa, Artificial Sequence
  The sequence is synthesized.
QQSYSTPYS

SEQ ID NO: 22 - protein, 11 aa, Artificial Sequence
  The sequence is synthesized.
RASQDIGSRLA

SEQ ID NO: 23 - protein, 7 aa, Artificial Sequence
  The sequence is synthesized.
AASSLQS

SEQ ID NO: 24 - protein, 10 aa, Artificial Sequence
  The sequence is synthesized.
QQYNSFSPIT

SEQ ID NO: 25 - protein, 11 aa, Artificial Sequence
  The sequence is synthesized.
RASQDIGSRLA

SEQ ID NO: 26 - protein, 7 aa, Artificial Sequence
  The sequence is synthesized.
AASSLQS

SEQ ID NO: 27 - protein, 10 aa, Artificial Sequence
  The sequence is synthesized.
QQYNSFSPIT

SEQ ID NO: 28 - protein, 11 aa, Artificial Sequence
  The sequence is synthesized.
RASQGIGSRLA

SEQ ID NO: 29 - protein, 7 aa, Artificial Sequence
  The sequence is synthesized.
AASSLQS

SEQ ID NO: 30 - protein, 10 aa, Artificial Sequence
  The sequence is synthesized.
QQYNSFSPIT

SEQ ID NO: 31 - protein, 5 aa, Artificial Sequence
  The sequence is synthesized.
DYEMH

SEQ ID NO: 32 - protein, 16 aa, Artificial Sequence
  The sequence is synthesized.
ALDPKTGDTAYSQKFK

SEQ ID NO: 33 - protein, 6 aa, Artificial Sequence
  The sequence is synthesized.
FYSYTY

SEQ ID NO: 34 - protein, 16 aa, Artificial Sequence
  The sequence is synthesized.
RSSQSLVHSNANTYLH

SEQ ID NO: 35 - protein, 7 aa, Artificial Sequence
  The sequence is synthesized.
KVSNRFS

SEQ ID NO: 36 - protein, 9 aa, Artificial Sequence
  The sequence is synthesized.
SQNTHVPPT

SEQ ID NO: 37 - protein, 115 aa, Artificial Sequence
  The sequence is synthesized.
QVQLQESGPGLVKPSETLSLTCTVSGDSINYHYWSWIRQPPGKGLEWIGYIYYSGSTTYNPSLKSRVTMSVDPSKNHFSLKLTSVTAADTALYFCARQRGAYDYWGQGTLVTVSS

SEQ ID NO: 38 - protein, 115 aa, Artificial Sequence
  The sequence is synthesized.
QVQLQQSGPGLVKPSETLSLTCTVSGDSINYHYWSWIRQPPGKGLEWIGYIYYSGSTTYNPSLKSRVTMSVDPSKNHFSLKLTSVTAADTALYFCARQRGAYDYWGQGTQVTVSS

SEQ ID NO: 39 - protein, 115 aa, Artificial Sequence
  The sequence is synthesized.
QVQLQQSGPGLVKPSETLSLTCTVSGDSINYHYWSWIRQPPGKGLEWIGYIYYSGSTTYNPSLKSRVTMSVDPSKNHFSLKLTSVTAADTALYFCARQRGAYDYWGQGTLVTVSS

SEQ ID NO: 40 - protein, 115 aa, Artificial Sequence; identical to SEQ ID NO: 39
  The sequence is synthesized.

SEQ ID NO: 41 - protein, 115 aa, Artificial Sequence; identical to SEQ ID NO: 39
  The sequence is synthesized.

SEQ ID NO: 42 - protein, 107 aa, Artificial Sequence
  The sequence is synthesized.
DIQLTQSPSSVSASVGDRVTIACRASQDIGSRLVWYQQRPGKAPKLLIYAASSLQSGVPSRFSGSGSGTDFTLTISSLQPEDFATYYCQQSYSTPYSFGQGTKLEIK

SEQ ID NO: 43 - protein, 107 aa, Artificial Sequence; identical to SEQ ID NO: 42
  The sequence is synthesized.

SEQ ID NO: 44 - protein, 108 aa, Artificial Sequence
  The sequence is synthesized.
DIVMTQSPSSVSASVGDRVTITCRASQDIGSRLAWYQQKPGKAPQLLIYAASSLQSGVPSRFSGSGSGTEFTLTISSLQPDDFATYYCQQYNSFSPITFGQGTRLEIK

SEQ ID NO: 45 - protein, 108 aa, Artificial Sequence
  The sequence is synthesized.
DIVMTQSPSSVSASVGDRVTITCRASQDIGSRLAWYQQKPGKAPKLLIYAASSLQSGVPSRFSGSGSGTDFTLTISSLQPEDFATYYCQQYNSFSPITFGQGTRLEIK

SEQ ID NO: 46 - protein, 108 aa, Artificial Sequence
  The sequence is synthesized.
DIVMTQSPSSVSASVGDRVTITCRASQGIGSRLAWYQQKPGKAPQLLIYAASSLQSGVPSRFSGSGSGTEFTLTISSLQPDDFATYYCQQYNSFSPITFGQGTRLEIK

SEQ ID NO: 47 - protein, 115 aa, Artificial Sequence
  The sequence is synthesized.
QVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 48 - protein, 112 aa, Artificial Sequence
  The sequence is synthesized.
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIK

SEQ ID NO: 49 - protein, 445 aa, Artificial Sequence
  The sequence is synthesized.
QVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 219 aa, Artificial Sequence
  The sequence is synthesized.
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 449 aa, Artificial Sequence
  The sequence is synthesized.
QVQLQESGPGLVKPSETLSLTCTVSGDSINYHYWSWIRQPPGKGLEWIGYIYYSGSTTYNPSLKSRVTMSVDPSKNHFSLKLTSVTAADTALYFCARQRGAYDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - protein, 212 aa, Artificial Sequence
  The sequence is synthesized.
DIQLTQSPSSVSASVGDRVTIACRASQDIGSRLVWYQQRPGKAPKLLIYAASSLQSGVPSRFSGSGSGTDFTLTISSLQPEDFATYYCQQSYSTPYSFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSC